(19) United States
(12) Patent Application Publication
(10) Pub. No.: US 2024/0352423 A1
(43) Pub. Date: Oct. 24, 2024

(54) METHODS AND SYSTEMS OF PRODUCING PRODUCTS SUCH AS ANIMAL DERIVED PRODUCTS

(71) Applicant: Terasaki Institute for Biomedical Innovation, Los Angeles, CA (US)

(72) Inventors: Seyedvahid Hosseini, Newbury Park, CA (US); Alireza Khademhosseini, Los Angeles, CA (US)

(73) Assignee: Terasaki Institute for Biomedical Innovation, Los Angeles, CA (US)

(21) Appl. No.: 18/281,034

(22) PCT Filed: Mar. 9, 2022
(86) PCT No.: PCT/US2022/019631

§ 371 Date: Sep. 8, 2023

Related U.S. Application Data
(60) Provisional application No. 63/314,171, filed on Feb. 25, 2022.
(60) Provisional application No. 63/314,191, filed on Feb. 25, 2022.
(60) Provisional application No. 63/300,577, filed on Jan. 18, 2022.
(60) Provisional application No. 63/279,617, filed on Nov. 15, 2021.
(60) Provisional application No. 63/279,631, filed on Nov. 15, 2021.
(60) Provisional application No. 63/279,642, filed on Nov. 15, 2021.
(60) Provisional application No. 63/279,644, filed on Nov. 15, 2021.
(60) Provisional application No. 63/164,397, filed on Mar. 22, 2021.
(60) Provisional application No. 63/164,387, filed on Mar. 22, 2021.
(60) Provisional application No. 63/159,403, filed on Mar. 10, 2021.

(51) Int. Cl. C12N 5/077; A23L 13/00

(52) U.S. Cl. CPC C12N 5/0658; A23L 13/00; C12N 2502/115

(57) ABSTRACT

Various systems and methods of producing animal derived products, such as for cultivated meat, are described herein. For example, one embodiment includes the steps of obtaining components of a cell-culture medium by sustainably harvesting blood components from living animals to produce a cell-culture medium suitable for in vitro cell culture and culturing cells in the cell-culture medium to produce a cultivated meat product. In some embodiments, the disclosure generally relates to sustainably harvesting blood components for the cultivation of meat and other animal derived products, e.g., by repeatedly extracting blood components in a manner which does not kill a non-human animal. In some embodiments, the non-human animals can be “farmed” for the purpose of sustainably extracting whole blood and/or blood products. In some embodiments, the harvested blood product may be used to produce a cultivated meat product or other animal derived product, such as a muscle tissue, a fat tissue or heme.

### RELATED APPLICATIONS

This application claims the benefit of U.S. Provisional Patent Application Ser. No. 63/159,403, filed Mar. 10, 2021, entitled “Constructs for Meat Cultivation and Other Applications”; U.S. Provisional Patent Application Ser. No. 63/279,617, filed Nov. 15, 2021, entitled “Constructs Comprising Fibrin or Other Blood Products for Meat Cultivation and Other Applications”; U.S. Provisional Patent Application Ser. No. 63/279,631, filed Nov. 15, 2021, entitled, “Methods and Systems of Preparing Cultivated Meat from Blood or Cellular Biomass”; U.S. Provisional Patent Application Ser. No. 63/279,642, filed Nov. 15, 2021, entitled, “Systems and Methods of Producing Fat Tissue for Cell-Based Meat Products”; U.S. Provisional Patent Application Ser. No. 63/279,644, filed Nov. 15, 2021, entitled “Production of Heme for Cell-Based Meat Products”; US Provisional Patent Application Serial No. U.S. 63/300,577, filed Jan. 18, 2022, entitled “Animal-Derived Antimicrobial Systems and Methods”; U.S. Provisional Patent Application Ser. No. 63/164,397, filed Mar. 22, 2021, entitled “Growth Factor for Laboratory Grown Meat”; U.S. Provisional Patent Application Ser. No. 63/164,387, filed Mar. 22, 2021, entitled, “Methods of Producing Animal Derived Products”; U.S. Provisional Patent Application Ser. No. 63/314,171, filed Feb. 25, 2022, entitled “Growth Factors for Laboratory Grown Meat and Other Applications”; and U.S. Provisional Patent Application Ser. No. 63/314,191, filed Feb. 25, 2022, entitled “Methods and Systems of Producing Products Such as Animal Derived Products.” Each of these is incorporated herein by reference in its entirety.

### FIELD OF THE INVENTION

The present disclosure relates in some aspects to sustainable methods of carrying out cell culturing generally, including to the production of cultivated meat and other animal derived products using farm animal blood derivatives sustainably obtained from live animals.

### BACKGROUND

Global production and consumption of meat continue to surge as demand is driven upward by population growth, individual economic gain, and urbanization. In 2012, the Food and Agriculture Organization (FAO) of the United Nations projected the global demand for meat would reach 455M metric tons by 2050 (a 76% increase from 2005). Likewise, the global demand for fish is projected to reach 140M metric tons by 2050.

This rising demand is problematic as current methods of large-scale animal husbandry are linked to public health complications, environmental degradation and animal welfare concerns. With regard to human health, the animal agriculture industry is interconnected with foodborne illness, diet-related disease, antibiotic resistance, and infectious disease. Notably, zoonotic diseases (e.g., Nipah virus, influenza A) are linked to agricultural intensification. Animal agriculture also contributes to environmental issues including greenhouse gas emissions, land use, and water use. The United Nations Intergovernmental Panel on Climate Change released a 2018 report asserting that greenhouse gas emissions must be reduced 45% by 2030 to prevent global temperatures from increasing 1.5° C.; a target that could mitigate catastrophes associated with a 2.0° C. increase. Conventional mitigation techniques include improvements in reforestation, soil conservation, waste management as well as tax policy, subsidies, and zoning regulations. While these strategies remain important, the urgency of climate change may require more transformative approaches. Lastly, with regard to animal welfare concerns, each year billions of animals are killed or suffer either directly (e.g., farm animal slaughter, seafood fishing) or indirectly (e.g., fishing by-catch, wildlife declines due to habitat destruction) in relation to human food systems.

The majority of the aforementioned issues can be attributed to the fact that the raw material inputs (i.e., animals) for conventional meat production are inherently unsanitary, inefficient, and sentient. Growing livestock and other animals consumed for food to maturity so that they can be slaughtered to provide meat is a time consuming and expensive process. As will be discussed in the present disclosure, by removing animals from the manufacturing process, several externalities may be alleviated.

Cell-based cultivated meat is a recent innovation in the food industry. Cell-based meat is manufactured using animal cells in vitro and in culture medium to create a meat without raising and slaughtering animals in traditional ways. As used herein cell-based meat is synonymous with cultivated meat, cultured meat, cellular meat, slaughter-free meat, and synthetic meat, among other related terms.

A significant amount of research has been done on variety of cell-based meat from different species of animals including mammals, fish and insects. It is projected that by 2030, the world population shall have surpassed 8.5 billion people. This projection will require a surplus of food production to suffice. The United States Department of Agriculture has estimated that 450 million tons of animal waste is produced annually.

Cultivated meat or cell-based meat is an alternative source of meat to replace animal-based meat. Cell-based meat is projected to be common in the global market in a few years, although one of major challenge is the high-cost associated with production of cultivated meat. Unfortunately, the economics of cell-based meat production are problematic with respect to large scale commercialization. For example, the beef burger cultivated by Maastricht University in 2013 is reported to have cost $280,400 ($2,470,000/kg) to produce. The production process involved three researchers using bench-scale techniques to expand 20,000 muscle cells over three months and served as a proof-of-concept rather than an attempt to scale production. A few groups have performed preliminary economic analyses to project the cost of cell-based meat for large-scale production scenarios. In 2008, The In Vitro Meat Consortium estimated, by modeling capital and growth medium costs based on data for single-cell protein production, cell-based meat could cost approximately twice as much as conventionally raised chicken. In 2014, a study speculating on the technical, societal, and economic factors of village-scale cell-based meat production calculated a cost range of $11-$520/kg dependent on the price of growth medium. Selected companies are targeting high-value products (e.g., foie gras, bluefin tuna, kangaroo meat) in order to lower the bar for reaching price parity.

The economics of cell-based meat production could be improved by decreasing the cost of the growth media. The most expensive component of current growth media is fetal bovine serum (FBS), which contains growth factors to stimulate proliferation of the cultivated cells. However, FBS is obtained from a bovine fetus, via a closed collection system, such as at a slaughterhouse, and was not intended to be manufactured in a sustainable or cost-effective way. To overcome this issue, and the aforementioned issues associated with large-scale animal husbandry, the cultivated meat field has excluded animals and animal products from the production of cell-based meats. For example, the field has taught methods to make cells grow in the absence of animal-derived growth factors by genetically manipulating the cells genome (see for example, US2019/024079A1, U.S. Ser. No. 10/920,196B2, or EP3638777A1), artificially supplementing the growth media with recombinant growth factors, transfecting the cultured cells with polynucleotides, such as messenger RNA, to transiently express the growth factors of interest during cultivation, or adding activators to the cell culture medium that stimulate production of specific growth factors, such as basic fibroblast growth factor.

There remains a need in the art for a cell culturing media composition for production of cell-based meat capable of providing nutrition including proteins, peptides, vitamins, cytokines and growth factors such as are present in animal blood. There thus remains a need in the art for sustainable methods for providing blood products while minimizing the necessity for slaughtering those animals. As disclosed herein, some embodiments are directed to a variety of systems and methods to harvest growth factors, proteins, vitamins, and the like from animals that are farmed for their blood and/or blood components, and not slaughtered for their meat.

### SUMMARY

In some embodiments, an economically viable process is provided in which animals are farmed to extract blood components, and not slaughtered for their meat. Blood components sustainably harvested from such animals may then be used as nutrients in cell culture media to supporting cell proliferation, as the building blocks for fabricating microfibrillar scaffolds necessary to produce texturized meat, and as a coloring additive, e.g., heme to impart the meat with a red color and to improve taste, etc. Blood components can be harvested in certain embodiments from living animals in a sustainable manner and with minimal ethical burdens. Accordingly, sustainable methods are provided of using animals to supply cell culture media in some embodiments, and to aid in the cost-effective mass production of cell-based cultivated meat without animal scarification or abuses in certain cases.

Provided in some embodiments are methods of growing cells in a cell culture generally, including, in certain cases, producing animal derived products such as cultured meat products comprising the steps of: obtaining components of a cell-culture medium by harvesting blood components from living animals to produce a cell-culture medium suitable for in vitro cell culture and culturing cells in said in the cell-culture medium. The methods may be used to produce animal derived products including those such as fibrin containing texturized meat, products which comprise a fatty-like tissue as well as those containing harvested red blood cells (RBCs) and their components. While the blood components used in the methods can be harvested from humans the living animals are generally non-human animals and those such as typical farm animals which are raised specifically for the purpose of providing blood products for harvesting. Also provided are the products of such methods including but not limited to cultured meat products.

As used herein “harvested” is intended to refer to methods by which blood components are sustainably removed from animals without killing the animal such as by apheresis wherein blood is taken from an animal and selected products may be removed, e.g., via centrifugation, filtering, or other method, and some or all of the unseparated portion of the blood is returned to the animal. In this manner, products such as blood platelets can be obtained from the animal on a periodic basis, in accordance with certain embodiments, without slaughtering the animal in the same way that dairy cows are milked on a regular basis. While blood components such as red blood cells (RBCs) can usually only be harvested every eight weeks or so, blood platelets can be harvested by apheresis on a basis close to weekly. Accordingly, the blood products can be harvested from animals on cycles lasting between 3 and 30 days, and in some embodiments, for certain components, in cycles lasting between 3 and 10 days. In particular, plasma and platelets can be harvested as frequently as twice per week while red blood cells can generally only be harvested as frequently as once a month. Thus, animals can be raised “farmed” for the specific purpose of being sources of blood components used as nutrient components for growing cells in cell culture. In some embodiments, methods by which farm animals can be raised for multiple purposes including for harvesting of blood products such cows, sheep and goats producing milk and sheep producing wool are described.

While blood components can be harvested from virtually and animal having blood, the blood components are generally harvested from species selected from the group consisting of cow, sheep, pig or swine, bison, elephant, whale, horse, deer, goat, and camels. While blood components can theoretically be harvested from a wide variety of other species including poultry and fish, doing so is generally impractical without slaughtering.

While some aspects of the present disclosure relate to the sustainable harvesting of blood products from non-human animals it is contemplated that additional blood and blood components obtained from slaughtered animals can be used to supplement the nutrient components sustainably harvested.

In some embodiments, blood components may be harvested by apheresis or other methods, for example, by using gravity to separate cells, such as centrifugation or sedimentation of red blood cells at atmospheric or high pressure, as known by those of skilled in the art, where red and white blood cells may be removed from a living animal and plasma and other blood components including platelets, red blood cells, fibrin and other proteins and blood components including platelet and other plasma soluble factors such as fibrin, albumin, minerals, vitamins and growth factors may be removed before the remaining blood product is returned to the animal. Such blood derivatives can thereby be obtained from living animals with minimal ethical burden and in a manner similar to that by which sera is regularly obtained from humans. Examples of media include those disclosed in U.S. Ser. No. 63/164,397 filed Mar. 22, 2021, the disclosure of which is hereby incorporated by reference.

In some embodiments, any desired blood component can be harvested by the present method, including platelet rich plasma (PRP) and platelets which can be added to a cell culture. In some embodiments, extracted platelets can be lysed by freeze-thawing process or physical shearing such as sonication to release their content including cytokines and growth factors. Extracted platelets can also be treated by physical, chemical or biochemical treatment to release cytokines and growth factors.

Suitable chemical and biochemical treatments include but are not limited to treatment with citrate, EDTA, calcium chloride, plasminogen activating factor and/or thrombin and suitable physical treatments include those selected from the group consisting of agitation, aging and adhesion of platelets to surfaces.

In some embodiments, blood components used in the methods include those selected from the group consisting of protein, peptides, vitamins, cytokines and growth factors as well as those selected from the group consisting of peptides, vitamins, cytokines and growth factors; synthetic and/or recombinant proteins, peptides, vitamins, cytokines and growth factors are added to the cell-culture medium to boost the proliferation and differentiation of cells are also contemplated in some embodiments.

In some embodiments, the cells cultured according to the methods described herein may be cultured to produce a member selected from the group consisting of cell-based meat, organs, skin, tusks, and horns with the demand for cell-based meat contemplated to be substantial. The cultured cells are therefore selected from the group consisting of myoblasts, fibroblasts, adipocyte, vascular, osteoblasts, tenocyte epithelial cells, mammalian glands, and neural cells as well as stem cells such as mesenchymal stem cells and induced pluripotent stem cell isolated from vertebrate and invertebrate animals from species selected from the group consisting of cow, sheep, swine, elephant, horse, deer, goat, camel, felines, canines, poultry, fish, crab, shrimps, bison, crocodile, and insects. It is also contemplated that analogues of exotic animal tissues such as tiger liver and rhinoceros' horn from threatened species can be produced in sustainable and cruelty-free ways.

In some embodiments, the methods as described herein can be carried out using blood components extracted from the blood of one species and used for cultivation of cells of the same species, or of different species and where blood products extracted from the blood of multiple species are used to provide nutrition to multiple cells.

In addition, various systems and methods of producing animal derived products, such as for cultivated meat, are described herein. For example, one embodiment includes the steps of obtaining components of a cell-culture medium by sustainably harvesting blood components from living animals to produce a cell-culture medium suitable for in vitro cell culture and culturing cells in the cell-culture medium to produce a cultivated meat product. In some embodiments, the disclosure generally relates to sustainably harvesting blood components for the cultivation of meat and other animal derived products, e.g., by repeatedly extracting blood components in a manner which does not kill a non-human animal. In some embodiments, the non-human animals can be “farmed” for the purpose of sustainably extracting whole blood and/or blood products. In some embodiments, the harvested blood product may be used to produce a cultivated meat product or other animal derived product, such as a muscle tissue, a fat tissue or heme.

In some embodiments, the disclosure is directed toward one or more methods of producing a cultivated meat or cultivated product, using for example, non-human animal cells.

In some embodiments, the methods comprise producing a plurality of meat products from one or more blood components extracted from the blood of non-human living animals, wherein the mass of the meat products are at least 110% of the mass of the non-human animals' meat after slaughtering.

In some embodiments, the methods comprise cultivating at least 0.1 kg of tissue/L platelet-rich plasma obtained from a first blood draw drawn from a non-human living animal and cultivating at least 0.1 kg of tissue/L platelet-rich plasma obtained from a second blood draw drawn from the non-human living animal.

In some embodiments, the methods comprise cultivating at least 0.1 kg of tissue/L plasma obtained from a first blood draw drawn from a non-human living animal and cultivating at least 0.1 kg of tissue/L plasma obtained from a second blood draw drawn from the non-human living animal.

In some embodiments, the methods comprise withdrawing a first blood draw having a volume of at least 1 mL/kg of body weight from a non-human living animal, cultivating at least 0.1 kg of tissue/L of volume obtained from the first blood draw, withdrawing a second blood draw having a volume of at least 1 mL/kg of body weight from the living animal, and cultivating at least 0.1 kg of tissue/L of volume obtained from the second blood draw.

In some embodiments, the methods comprise withdrawing a plurality of blood draws, wherein each blood draw has a volume of at least 1 mL/kg of body weight from the living animal and cultivating at least 0.1 kg of tissue/L of volume obtained from each blood draw.

In some embodiments, the methods comprise withdrawing a plurality of blood draws from a non-human living animal without killing the animal and forming a plurality of cultivated meat products using the plurality of blood draws.

In some embodiments, the methods comprise withdrawing a plurality of blood draws from a non-human living animal over a period of time of at least 2 weeks and forming a plurality of cultivated meat products using the plurality of blood draws.

In some embodiments, the methods comprise raising a non-human living animal on a regenerative farm, withdrawing a plurality of blood draws from the living animal, and forming a plurality of cultivated meat products using the plurality of blood draws.

In some embodiments, the methods comprise raising a non-human living animal on a regenerative farm, harvesting at least one blood product and at least one other product from the non-human living animal, and using the at least one blood product to cultivate a cell-based meat.

In some embodiments, the method comprises grazing a non-human animal on a plurality of pastures such that the non-human animal increases carbon content of each of the pastures by at least 50 mass % due to its waste, relative to identical pastures in the absence of the non-human animal, withdrawing a plurality of blood draws from the non-human animal, and using the blood draws to produce a cell-based meat product.

In some embodiments, the methods comprise harvesting whole blood from a non-human animal, separating the whole blood into blood components, and using the blood components to produce a cell-based meat.

In some embodiments, the methods comprise harvesting at least one blood component from a non-human living animal using apheresis and using the at least one blood component to produce a cell-based meat product.

In some embodiments, the methods comprise producing a cultivated product from a non-human living animal blood derivatives wherein the mass of cultivated and animal derived product is at least 110% of the mass of the non-human animal product after slaughtering.

In some embodiments, the methods comprise withdrawing a plurality of blood draws from a non-human living animal without killing the animal and forming a cultivated product using one or more of the plurality of blood draws.

In some embodiments, the methods comprise producing animal derived products comprising the steps of obtaining blood component of a cell-culture growth medium by harvesting blood components from non-human living animals to produce a cell-culture growth medium suitable for in vitro cell culture, and simultaneously using the blood components to produce an animal derived product.

In some embodiments, the methods comprise producing animal derived products comprising the steps of obtaining growth supplement of a cell-culture growth medium by harvesting blood components from living animals to produce a cell-culture growth medium suitable for in vitro cell culture and culturing cells in said cell-culture growth medium.

In another aspect, the present disclosure encompasses methods of making one or more of the embodiments described herein, for example, a cultivated meat product. In still another aspect, the present disclosure encompasses methods of using one or more of the embodiments described herein, for example, to produce a cell-culture growth medium.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates the effect of platelet-rich plasma isolated from different cows on the proliferation of bovine myocytes, according to some embodiments;

FIGS. 2A-E illustrate the effect of varying the concentration of platelet-rich plasma, isolated from various cows, in the culture media on bovine myoblast proliferation. The platelet rich plasma was isolated from (FIG. 2A) cow 2371, (FIG. 2B) cow 4321, (FIG. 2C) cow 4266, (FIG. 2D) cow 4348, and (FIG. 2E) cow 14583, according to some embodiments;

FIGS. 3A-F illustrate the effect of varying the concentration of platelet-rich plasma, isolated from various cows, in the culture media on human hepatocyte proliferation. The platelet rich plasma was isolated from (FIG. 3A) cow 2398, (FIG. 3B) cow 14347, (FIG. 3C) cow 4266, (FIG. 3D) cow 2371, (FIG. 3E) cow 4321, and (FIG. 3F) cow 14424, according to some embodiments;

FIGS. 4A-H illustrate the hematology and blood chemistry results from cow 4266, including: (FIG. 4A) red blood cells (M/microliter), (FIG. 4B) hemoglobin (g/dL), (FIG. 4C) white blood cells (K/microliter), (FIG. 4D) platelets (K/microliter), (FIG. 4E) fibrinogen (mg/dL), (FIG. 4F) albumin (g/dL), (FIG. 4G) aspartate aminotransferase (U/L), and (FIG. 4H) alkaline phosphatase (U/L), according to some embodiments;

FIGS. 5A-H illustrate the hematology and blood chemistry results from cow 4348, including: (FIG. 5A) red blood cells (M/microliter), (FIG. 5B) hemoglobin (g/dL), (FIG. 5C) white blood cells (K/microliter), (FIG. 5D) platelets (K/microliter), (FIG. 5E) fibrinogen (mg/dL), (FIG. 5F) albumin (g/dL), (FIG. 5G) aspartate aminotransferase (U/L), and (FIG. 5H) alkaline phosphatase (U/L), according to some embodiments;

FIGS. 6A-H illustrate the hematology and blood chemistry results from cow 2315, including: (FIG. 6A) red blood cells (M/microliter), (FIG. 6B) hemoglobin (g/dL), (FIG. 6C) white blood cells (K/microliter), (FIG. 33D) platelets (K/microliter), (FIG. 6E) fibrinogen (mg/dL), (FIG. 6F) albumin (g/dL), (FIG. 6G) aspartate aminotransferase (U/L), and (FIG. 6H) alkaline phosphatase (U/L), according to some embodiments;

FIGS. 7A-H illustrate the hematology and blood chemistry results from cow 2371, including: (FIG. 7A) red blood cells (M/microliter), (FIG. 7B) hemoglobin (g/dL), (FIG. 7C) white blood cells (K/microliter), (FIG. 7D) platelets (K/microliter), (FIG. 7E) fibrinogen (mg/dL), (FIG. 7F) albumin (g/dL), (FIG. 7G) aspartate aminotransferase (U/L), and (FIG. 7H) alkaline phosphatase (U/L), according to some embodiments;

FIGS. 8A-H illustrate the hematology and blood chemistry results from cow 5211, including: (FIG. 8A) red blood cells (M/microliter), (FIG. 8B) hemoglobin (g/dL), (FIG. 8C) white blood cells (K/microliter), (FIG. 8D) platelets (K/microliter), (FIG. 8E) fibrinogen (mg/dL), (FIG. 8F) albumin (g/dL), (FIG. 35G) aspartate aminotransferase (U/L), and (FIG. 8H) alkaline phosphatase (U/L), according to some embodiments;

FIGS. 9A-H illustrate the hematology and blood chemistry results from cow 5276, including: (FIG. 9A) red blood cells (M/microliter), (FIG. 9B) hemoglobin (g/dL), (FIG. 9C) white blood cells (K/microliter), (FIG. 9D) platelets (K/microliter), (FIG. 9E) fibrinogen (mg/dL), (FIG. 9F) albumin (g/dL), (FIG. 9G) aspartate aminotransferase (U/L), and (FIG. 9H) alkaline phosphatase (U/L), according to some embodiments;

FIGS. 10A-H illustrate the hematology and blood chemistry results from cow 14424, including: (FIG. 10A) red blood cells (M/microliter), (FIG. 10B) hemoglobin (g/dL), (FIG. 10C) white blood cells (K/microliter), (FIG. 10D) platelets (K/microliter), (FIG. 10E) fibrinogen (mg/dL), (FIG. 10F) albumin (g/dL), (FIG. 10G) aspartate aminotransferase (U/L), and (FIG. 10H) alkaline phosphatase (U/L), according to some embodiments; and

FIGS. 11A-H illustrate the hematology and blood chemistry results from cow 14583, including: (FIG. 11A) red blood cells (M/microliter), (FIG. 11B) hemoglobin (g/dL), (FIG. 11C) white blood cells (K/microliter), (FIG. 11D) platelets (K/microliter), (FIG. 11E) fibrinogen (mg/dL), (FIG. 38F) albumin (g/dL), (FIG. 11G) aspartate aminotransferase (U/L), and (FIG. 11H) alkaline phosphatase (U/L), according to some embodiments.

### DETAILED DESCRIPTION

The present disclosure generally relates to “sustainably harvesting blood components” for the cultivation of meat and other animal derived products, by repeatedly extracting blood components in a manner which does not kill a non-human animal. In some embodiments, the non-human animals can be “farmed” for the purpose of sustainably extracting whole blood and/or blood products. It should be understood by those skilled in the art, that in some cases, the non-living animal may be used to produce one or more products, such as for example, blood and wool. For instance, in some embodiments, a cow may be used to produce blood products and/or milk, whereas a sheep may be used to produce blood products and/or wool.

Some embodiments are directed toward repeated blood collection from non-human animals, for example, to obtain whole blood or blood components, etc. For example, in some embodiments, blood may be withdrawn from the animal at spaced intervals, so as to allow the animal time to recover and produce new blood. For instance, blood may be withdrawn from the animal every 2 weeks, every 4 weeks, every 6 weeks, every 2 months, or the like. The blood draws may be processed, for example, as discussed herein. For example, the blood may be used to obtain platelet rich plasma to stimulate cell growth in a bioreactor, e.g., as discussed herein. In this way, such cells can be obtained in certain embodiments in a sustainable and cost-effective manner, e.g., without killing the animal. This usage may result, in certain embodiments, in the reduction in carbon emissions, water use, land use, etc.

For example, in some embodiments, apheresis can be used to remove one or more blood components, such as for example, red blood cells, platelets, plasma, etc., and to return unharvested blood components back to the non-human animal. In other embodiments, whole blood can be harvested and separated, for example, into blood components, using any technique known to those of ordinary skill, for example, centrifugation or sedimentation. Other embodiments are directed toward the use of plasmapheresis (i.e., isolation of plasma product), erythrocytapheresis (i.e., isolation of red blood cells), plateletpheresis (i.e., isolation of platelets), leukapheresis (i.e., isolation of white blood cells) to isolate the desired blood products.

The above discussions are non-limiting examples of certain aspects generally directed to sustainably harvesting blood products from non-human animals for the cultivation of meat or other products. However, other embodiments are also possible besides those discussed above.

For example, certain aspects are generally directed to cultivated animal-derived products, such as cultivated meat, or other products. These may be produced, for example, using cells taken from an animal, but then the cells are cultured in vitro, e.g., using bioreactors, flasks, petri dishes, microwell plates, or other cell culture systems. Many cell culture systems will be known to those of ordinary skill in the art. This is in stark contrast to traditional techniques of sacrificing animals and harvesting their meat or other organs (e.g., skin, internal organs, etc.) for food or other uses. Although the original cells seeded to form the product may have originated or otherwise have originally been derived from a living animal, the bulk of the cells forming the actual product were grown or cultured in an in vitro setting, rather than naturally as part a living animal.

A variety of products may be formed from cells cultured in vitro. For instance, in certain embodiments, the products may form “cultivated meat,” or meat that is intended to be eaten, for example, by humans. It will be appreciated that, because it is to be eaten, such products will often be formed of edible or digestible materials, e.g., materials that can be digested, or degraded to form generally nontoxic materials within the digestive system. For instance, the cultivated meat may contain animal-derived cells (e.g., derived from a chicken, a cow, a pig, a sheep, a goat, a deer, a fish, a duck, a turkey, a shrimp, a bison, a whale, an elephant, or other animals that are commonly recognized for widespread human consumption), such as muscle cells, fat cells, or the like. The cells may be wild-type or naturally occurring cells (e.g., harvested from an animal), although in some embodiments, the cells may include genetically engineered cells, e.g., engineered in a way to increase proliferation. In addition, in some embodiments, the cultivated meat product may contain other edible materials, such as plant-originated materials. Non-limiting examples of edible materials include proteins, carbohydrates, sugars, saccharides, plant-based fats, etc., as well as polymers formed from these (for example, polylactic acid, polyglycolic acid, cellulose, etc.). In some cases, the edible materials may be digested to form nutrients, e.g., such as amino acids, sugars, etc. that have nutritional value, for example, when taken up into the body. However, in some cases, the edible materials cannot be digested, and/or can be digested to form non-nutrients that cannot be absorbed as nutrients but can be passed through the digestive system without detrimental effects.

In addition, it should be understood that the invention is not limited to only cultivated meat products. In some cases, products such as those described herein may be cultivated from animal-derived cells, but the product is not necessarily one that is intended to be eaten. For instance, cells from an animal may be cultured to form various organs that can be harvested, such as skin, hair, fur, or the like. Thus, as a non-limiting example, milk, leather, wool, cultivated fur, etc. can be formed by growing cells in culture, for example as discussed herein, without the traditional method of sacrificing animals to harvest their skin or other organs.

Some aspects of the disclosure are directed at sustainably procuring blood components for producing cultivated meat products or other products, for example, in a bioreactor. In some embodiments, blood can be collected from non-human animals, for example, to obtain platelets, platelet-rich plasma, white blood cells, red blood cells, etc. For example, repeated blood collection from non-human animals may be used to obtain immune cells, or the like.

As will be appreciated by those skilled in the art, certain animal derived blood products, such as platelets, red blood cells, etc., may be safely harvested from different animals such as bovines, equines and canines, and used to treat a wide range of injuries in veterinary medicine. Thus, in some embodiments, blood components, such as platelets and red blood cells, may be repeatedly harvested from animals, e.g., without causing health issues to the animals. For example, platelets and platelet rich plasma can be harvested at least 1×, at least 2×, at least 3×, at least 4×, etc., every 30 days, or at other rates such as those described herein. In this way, blood components may be sustainably harvested and used, for example, for the production of cultivated meat, e.g., cell culture media, without slaughtering the animal and/or without the adverse environmental impacts of raising animals for slaughter.

Accordingly, in one set of embodiments, one or more blood draws may be withdrawn or taken from a non-human living animal, e.g., to obtain platelet rich plasma (PRP) or red blood cells, e.g., including those described herein. Exemplary embodiments of non-human living animals include chicken, cow, pig, sheep, pig, horse, bison, elephant, camel, mutton, goat, deer, fish, duck, turkey, shrimp, or other animals that are commonly recognized for widespread human consumption. The blood may be processed in some cases to isolate the various components, e.g., platelet-rich plasma, red blood cells etc., using any suitable technique, e.g., centrifugation and apheresis.

Some aspects of the present disclosure are directed to enhancing cell proliferation using a platelet-rich plasma and/or platelet lysate, for example, in bioreactors containing products such as cultivated meat products, or other bioreactors or applications. For instance, some embodiments are directed toward activating cultures of platelet rich plasma and/or platelet concentrates to produce a serum solution comprising at least one growth factor. In some cases, the cells can be filtered out of the serum solution. This serum solution (with or without cells) can be used in a variety of applications. For instance, it may be used within the same or a different bioreactor to enhance cell adhesion and proliferation. Other examples of suitable applications include, but are not limited to, biopharmaceuticals, animal furs, cell-based organs, etc., which can be manufactured, for example, as discussed herein.

The serum solution may be prepared and used as discussed herein, e.g., relatively soon after preparation. However, in some embodiments, the serum solution can be stored for at least 1 week, at least 2 weeks, at least 1 month, at least 6 months, at least 12 months, etc. at room temperature or other temperatures, e.g., at 4° C., at 0° C., at −4° C., at −20° C., etc. As a non-limiting example, the serum solution may be freeze-dried in some embodiments. In addition, in some embodiments, the freeze-dried serum solution can be reconstituted, e.g., at its original concentration, or at higher or lower concentrations, such as at concentrations that are at least 10×, at least 50×, or at least 100× as concentrated as the original serum solution.

Certain aspects of the disclosure are directed toward obtaining growth factors and/or other components from various blood components, e.g., platelets, plasma, and/or platelet-rich plasma can be isolated in some embodiments directly from the blood of a non-human animal, e.g., cow, pig, sheep, goat, deer, fish, bison, whale, elephant, camel, duck, turkey, shrimp, etc.

Thus, platelet rich plasma (PRP) can be derived from whole blood from which red blood cells have been removed, such as by centrifugation. Plasma can also be derived from whole blood by, for example, using apheresis or by removing platelets from the platelet-rich plasma product using, for example, centrifugation. Plasma and platelet rich plasma (PRP) contain a variety of growth factors that are in blood such as transforming growth factor beta, fibroblast growth factor, insulin-like growth factor 1, insulin-like growth factor 2, vascular endothelial growth factor, epidermal growth factor, interleukin 8, keratinocyte growth factor and connective tissue growth factor. In addition, lysing the platelets in the platelet-rich plasma can release platelet-derived growth factor. Platelet rich plasma (PRP) can be categorized based on its leukocyte and fibrin content as leukocyte-rich PRP (L-PRP), leukocyte reduced PRP (P-PRP); leukocyte reduced or pure PRP, (4) leukocyte platelet-rich fibrin and pure platelet-rich fibrin (L-PRF). As used herein, “platelet-rich plasma” (“PRP”) is plasma having platelets at a concentration of at least 2×, at least 5×, or at least 10× the normal concentration of platelets in blood. A “platelet-poor plasma” is a plasma comprising some platelets, but at a concentration that is less than the normal concentration of platelets in blood.

Plasma and/or platelet-rich plasma contain growth factors, and other solutes, known by those skilled in the art, to enhance biomass production. For example, plasma and/or platelet rich plasma comprises adhesive proteins, e.g., fibrinogen, which can facilitate non-human cells, e.g., myoblasts, to adhere to microcarriers, e.g., fibrin microcarriers; it also comprises growth factors, e.g., platelet-derived growth factor that enhance cell proliferation. Other solutes of plasma and/or platelet-rich plasma include dissolved proteins (6-8% by weight), e.g., serum albumins, goblins, and fibrinogen), glucose, clotting factors, electrolytes (Na⁺, Ca²⁺, Mg²⁺, HCO₃⁻, Cl⁻, etc.), and hormones, etc. As such, in some embodiments, the plasma and/or platelet-rich plasma can be added to any cell culture media to produce a cell culture growth medium and to grow non-human cells, for example, in a bioreactor to produce a cell-based meat product. For example, in some cases plasma and/or platelet-rich plasma can be added to any cell basal culture media, such as DMEM, alpha-MEM, DMEM-F12 or basal Essential 8, to produce a cell culture growth medium, and added to a bioreactor, for example, containing a cell-based meat product.

In some instances, it may be desirable to obtain a platelet concentrate directly from whole blood, for example, by apheresis or from a platelet-rich plasma by, for example, centrifugation, or other techniques known to those of skill in the art, e.g., tangential flow filtration. In some embodiments, the final platelet concentration in platelet-rich plasma is at least 10⁵ platelets/mL, at least 10⁶ platelets/mL, at least 10⁷ platelets/mL, at least 10⁸ platelets/mL, at least 10⁹ platelets/mL, at least 10¹⁰ platelets/mL, etc., in the platelet concentrate.

As used herein “apheresis” refers to any process by which blood or blood products are removed from an animal and one or more components are separated from the blood or blood product with the remaining components returned to the animal's circulation. For example, apheresis machines can be used to remove select blood components, such as for example, platelets and/or plasma and to return unused blood components, e.g., red blood cells, back to the animal. In this way, plasma and platelets can be donated more frequently as they have a higher turnover than, for example, red blood cells. In some cases, whole blood samples can be withdrawn and preserved, for example, in blood bags (e.g., containing an anticoagulant), and processed, e.g., by centrifugation, to separate the platelet-rich plasma, immune cells (i.e., the buffy coat), red blood cells, etc.

In some embodiments, a method for producing a cultivated meat product comprising withdrawing a plurality of blood draws from a non-human living animal over a period of time and forming a plurality of cultivated meat products using the plurality of blood draws is provided. For example, in some cases, blood may be withdrawn from a donor animal at spaced intervals, which may be regular or irregular. Between blood draws, the animal can recover and produce new blood. Any suitable interval may be used. For example, the blood may be withdrawn from the animal every 2 weeks, every 4 weeks, every 6 weeks, every 2 months, or the like. In addition, in some cases, blood may be withdrawn from an animal at a period of greater than 1 day, greater than 2 days, greater than 3 days, greater than 4 days, greater than 5 days, greater than 6 days, greater than 1 week, greater than 2 weeks, greater than 3 weeks, greater than 4 weeks, greater than 5 weeks, greater than 6 weeks, greater than 7 weeks, greater than 8 weeks, greater than 9 weeks, greater than 10 weeks, greater than 11 weeks, greater than 12 weeks, greater than 13 weeks, greater than 14 weeks, greater than 15 weeks, greater than 16 weeks, etc. In certain embodiments, the period may be no more than 16 weeks, no more than 15 weeks, no more than 14 weeks, no more than 13 weeks, no more than 12 weeks, no more than 11 weeks, no more than 10 weeks, no more than 9 weeks, no more than 8 weeks, no more than 7 weeks, no more than 6 weeks, no more than 5 weeks, no more than 4 weeks, no more than 3 weeks, no more than 2 weeks, no more than 1 week, no more than 6 days, no more than 5 days, no more than 4 days, no more than 3 days, no more than 2 days, etc. Combinations of any of these are also possible. For example, the period may be between 3 weeks and 5 weeks, between 6 days and 2 weeks, or the like. As mentioned, in some case, the blood withdrawal may be relatively irregular, but the average period of withdraws may, in some embodiments, be within these ranges.

In some embodiments, the first blood draw and the second blood draw are separated by at least 24 hours. As an example, in some embodiments, a blood draw may be withdrawn from an animal, and after a suitable interval, e.g., 24 hours, an additional blood draw may be withdrawn from the animal. This cycle can also be repeated any suitable number of times. In addition, in some embodiments, the first blood draw and the second draw may be separated by at least 1 day, at least 2 days, at least 3 days, at least 4 days, at least 5 days, at least 6 days, at least 7 days, at least 14 days, at least 21 days, at least 28 days, at least 5 weeks, at least 6 weeks, at least 7 weeks, at least 8 weeks, etc. The blood draws may each be processed, for example, as discussed herein. For example, the blood may be used to isolate platelets and/or platelet-rich plasma.

In addition, the animal may also be subjected to second, third, etc. blood draws, e.g., at spaced intervals such as discussed herein. The blood withdrawn in each draw may be treated in the same way, or in different ways, depending on the application. For example, in some embodiments, a cell growth media may be produced using the cells, e.g., a platelet lysate, obtained from a third blood draw from the non-human living animal. In some embodiments, the animal remains alive between blood draws, and can recover and produce new blood. In this way, although blood production from the animal occurs, the animal is not slaughtered in order for blood production from the animal to continue.

For example, in one set of embodiments, blood components such as red blood cells (RBCs) may be harvested every eight weeks or so, blood platelets can be harvested by apheresis on a basis close to weekly. Accordingly, the blood products can be harvested from animals on cycles lasting between 3 and 30 days and for certain components in cycles lasting between 3 and 10 days. For example, plasma and platelets can be harvested as frequently as twice per week while red blood cells can be harvested as frequently as once a month. It should be noted that these are examples, and that in other embodiments, blood may be withdrawn from a donor animal at spaced intervals in order to harvest blood components such as red blood cells, platelets, plasma, etc. at different periods, e.g., any of the periods previously described.

Thus, in some embodiments, animals can be raised “farmed” for the purpose of being sources of blood components used as nutrient components for growing cells in cell culture. Contemplated in certain embodiments are methods by which farm animals can be raised for multiple purposes including, for example, for the harvesting of blood products such cows, sheep and goats producing milk and sheep producing wool. In some embodiments, the nutrient components may be used to produce cultivated meat or other animal derived products, e.g., as discussed herein.

Some embodiments are directed toward harvesting at least one blood component from a non-human living animal using apheresis and using the at least one blood component to produce a cell-based meat product. For example, in some embodiments blood products, e.g., platelet-rich plasma, may be extracted using apheresis from a blood draw and used to cultivate a tissue, e.g., a meat product. In some instances, at least 0.1 kg of tissue may be cultivated per liter of blood component, e.g., platelet-rich plasma or plasma, withdrawn from a first blood draw; in other instances, at least 0.1 kg of tissue may be cultivated per liter of blood component, e.g., platelet-rich plasma or plasma, withdrawn from a second blood draw. In some embodiments, the first blood draw comprises producing at least 50 mL/kg, at least 100 mL/kg, at least 200 mL/kg, at least 300 mL/kg, at least 400 mL/kg, at least 500 mL/kg, at least 600 mL/kg, or at least 700 mL/kg of blood component, e.g., platelet-rich plasma or plasma, from the first blood draw using apheresis. In some embodiments, the second blood draw comprises producing at least 50 mL/kg, at least 100 mL/kg, at least 200 mL/kg, at least 300 mL/kg, at least 400 mL/kg, at least 500 mL/kg, at least 600 mL/kg, or at least 700 mL/kg of blood component, e.g., platelet-rich plasma or plasma, from the second blood draw using apheresis. In some embodiments, a first blood draw having a volume of at least 10 mL/kg of body weight from a non-human living animal may be withdrawn from a non-human living animal and used to cultivate at least 0.1 kg of tissue, e.g., cultivated meat, per liter of volume obtained from the first blood draw. A second blood draw also having a volume of at least 10 mL/kg of body weight from a non-human living animal may also be withdrawn and used to cultivate at least 0.1 kg of tissue, e.g., a fatty-like tissue, per liter of volume obtained from the second blood draw. In some embodiments, the method comprising withdrawing a plurality of blood draw thereafter, for example, after a first and second blood draws, wherein each blood draw has a volume of at least 10 mL/kg of body weight from the living animal and cultivating at least 0.1 kg of tissue per liter of volume obtained from each blood draw.

In some embodiments, a cultivated meat product may be produced from a plurality of animals, wherein the mass of the cultivated meat product is at least 110%, at least 150%, at least 200%, at least 250%, at least 500%, at least 1000%, at least 1500%, or at least 1800% of the mass of the plurality of animals. In other words, according to certain embodiments, more tissue mass may ultimately be produced starting from the cells taken from the animal by cultivating cells as discussed herein, then the actual mass of the animal itself. In some embodiments, a cultivated meat product may be produced from a non-human living animal, wherein the mass of the cultivated meat product is at least 110% of the mass of the non-human living animal. In other embodiments, the mass of the cultivated meat product is at least 10%, at least 20%, at least 30%, at least 40%, at least 60%, or at least 70% of the mass of the single animal, e.g., where the cells used to produce the meat product originated.

In some embodiments, a blood draw is performed on a non-human living animal and used to produce a cultivated meat product from at least a portion of the blood draw. In some embodiments, the method comprises extracting a blood component from the blood draw using apheresis. For example, in some instances it may be desirable to withdraw a platelet-rich plasma from the blood draw by, for example, apheresis, and to use the platelet-rich plasma to cultivate cells in a bioreactor to produce a cultivated meat product. In some embodiments, the blood components to be extracted may be selected from the group consisting of a platelet concentrate, a platelet-rich plasma, a platelet-poor plasma, and a plasma product. At times, it may be desirable to manipulate one or more of the blood components to produce a new product. For example, in some embodiments, the method comprises freeze-thawing the platelet concentrate to produce a platelet-lysate. In other embodiments, the method comprises adding thrombin to the plasma product to crosslink the plasma and producing a serum product. In some embodiments, the method comprises adding one or more blood components to a bioreactor and cultivating cells within the bioreactor to produce a cultivated meat product.

For example, in some embodiments, the mass of the cultivated meat product produced in the bioreactor is at least 0.1 kg of the cultivated meat product per liter of the blood component, e.g., platelet-rich plasma, plasma, etc., or other masses such as those described herein. For example, the mass may be at least 0.2 kg, at least 0.3 kg, at least 0.5 kg, at least 1 kg, at least 1.5 kg, at least 2 kg, at least 2.5 kg, at least 3 kg, at least 4 kg, at least 5 kg, at least 6 kg, at least 7 kg, at least 8 kg, at least 9 kg, at least 10 kg, etc. of the cultivated meat product per liter of the blood component.

In some embodiments, the animals may be pregnant and/or nursing. Without wishing to be bound by theory, those of ordinary skill in the art will understand that maternal serum often comprises higher levels of hormones and growth factors that foster growth of the fetus during pregnancy and/or during nursing. Performing blood draws on animals that are pregnant and/or nursing in some cases may yield blood products with enhanced concentrations of such growth factors, such as for example, insulin-like growth factors, and/or may enhance the proliferation of animal cells during cultivation. In some embodiments, blood may be drawn from an animal at any point during its pregnancy. In other embodiments, blood may be drawn from an animal at any point during the lactation period.

As those of ordinary skill in the art will appreciate, the non-human animals of interest may have internal blood volumes between 50 mL/kg and 80 mL/kg of body weight. For example, a cow's blood volume is typically between about 52-57 mL/kg of body weight, a horse's blood volume is typically between about 70-80 mL/kg of body weight, a pig's blood volume is typically between about 60-70 mL/kg of body weight, and a sheep's blood volume is typically between about 55-65 mL/kg of body weight. In some embodiments, a first blood draw has a volume of between 0.1% and 5%, in liters per kg of weight of the non-human living animal. In other embodiments, a second blood draw has a volume of between 0.1% and 5%, in liters per kg of weight of the non-human living animal.

In some embodiments, the first and second blood draws have a volume of between 1% to 3%, in liters per kg of weight of the non-human living animal. Some non-limiting embodiments are generally directed to methods for withdrawing a plurality of blood draws, e.g., a first blood draw, from a non-human living animal without killing the animal and forming a plurality of cultivated meat products, e.g., a fatty-like tissue, using the plurality of blood draws. In some embodiments, the method comprises withdrawing a plurality of blood draws from a non-human living animal without killing the animal and forming a plurality of cultivated meat products using the plurality of blood draws. In other embodiments, the method comprises withdrawing a plurality of blood draws from a non-human living animal over a period of time of at least 12 weeks, or other periods such as those described herein. A blood draw may comprise withdrawing whole blood, or in some cases, one or more blood products commonly found in whole blood. Non-limiting examples of components commonly found in whole blood include red blood cells, platelets, blood plasma, white blood cells.

Blood plasma may further comprise water, dissolved proteins (e.g., serum albumins, globulins, fibrinogen, etc.), glucose, clotting factors, electrolytes (e.g., Na⁺, Ca²⁺, Mg²⁺, HCO³⁻, Cl⁻, etc.), hormones, carbon dioxide, and oxygen. In some embodiments, one or more blood products may be obtained by, for example, apheresis. Non-limiting examples of the blood products that may be obtained using apheresis include blood plasma (i.e., plasmapheresis), red blood cells (i.e., erythrocytapheresis), blood platelets (i.e., plateletpheresis), and white blood cells (i.e., leukapheresis) or any combination thereof (e.g., platelet-rich plasma). In some embodiments, apheresis is used to obtain platelet-rich plasma. For example, in some embodiments, producing the platelet-rich plasma from a first blood draw using apheresis comprises removing between 10 mL/kg and 15 mL/kg of platelet rich plasma from the first blood draw. In other embodiments, producing the platelet-rich plasma from a second blood draw using apheresis comprises removing between 10 mL/kg and 15 mL/kg of platelet rich plasma from the second blood draw. The cells obtained from the blood draws, e.g., platelets and/or platelet-rich plasma, can be used to create, for example, a cell culture media by lysing the platelets to produce a platelet lysate and adding it to a cell culture medium, e.g., DMEM. The platelet lysate-based cell culture media can be added to a cell culture system, e.g., a bioreactor, comprising non-human muscle, for example, to increase muscle mass. Animal and/or plant-derived oils can be dispersed within plasma isolated from the plurality of blood draws and polymerized to from a fatty-like tissue. Plasma comprises high concentrations of soluble fibrinogen, which upon addition of a clotting agent, e.g., thrombin, stimulates the cleavage of fibrinogen to fibrin, which self-assembles into a soft hydrogel, entrapping the dispersed oil phase within it. In other embodiments, red blood cells isolated from the plurality of blood draws may be lysed, e.g., by freeze thawing, to release heme and used as a natural meat colorant to impart redness to a cultivated meat product.

While some of the articles and/or methods described herein are directed to the sustainable production of cultivated meat products, and other cultivated animal-derived products, blood and blood products from, e.g., slaughterhouses producing traditional meat products, can also be used in other embodiments.

Some aspects of the disclosure are directed to raising a non-living animal blood donor on a farm, such as a regenerative farm. Herein, the term regenerative farming is synonymous with regenerative agriculture, agricultural farming, regenerative growers, regenerative grazing, and the like. As will be appreciated by those of skill in the art, the majority of the food consumed around the world is grown through varying methods of intensive industrial agriculture, a form of modern farming that became prevalent during the industrial revolution. However, industrial agriculture has been linked to problems such as (i) diminished biodiversity among plants and animals, (ii) soil, water, and air pollution, (iii) soil erosion, (iv) unsustainable rates of water consumption, (v) development of chronic disease, cancers, foodborne pathogens, pesticides, and high-speed meat production, (vi) antibiotic resistance, and (vii) a lack of nutritional value in food. Regenerative farming is a holistic approach to agriculture that focuses on the interconnection of farming systems and the ecological system as a whole.

Regenerative farming may involve the integration of animals into the farm as much as possible. For example, regenerative grazing is one practice of regenerative farming that builds soil health by managing livestock on perennial and annual forages. One outcome of current industrial agricultural practices is monocropping, in which a single crop, e.g., wheat, is grown on the same piece of land year after year. Such practices strip the soil of its nutrients and requires hauling manure at additional cost or importing synthetic fertilizers from off the farm to support continued growth of the crops. However, proper management of livestock using, for example, regenerative grazing practices, can provide the manure needed to replace nutrients to the soil, reducing the need for fertilizers, and increasing soil organic matter. For example, healthy soils capture large amounts of carbon and water and reduce the amount of polluted runoff.

In some cases, regenerative farming may involve practices of reusing and recycling frequently, e.g., wherever possible. As such, in some embodiments, a single animal can be used for multiple purposes, such as blood donation and milk production. For example, a cow can be used to donate bovine blood for producing cultivated meat products and milk; a sheep may also be used to donate blood for cultivated meat production and wool.

In some embodiments, regenerative farming may involve sustainability. In some embodiments, regenerative farming practices may lower the economic hurdles associated with large scale commercialization of cultivated meats by providing a cell growth media in a sustainable manner. Currently, fetal bovine serum is used as the standard growth supplement for most cell culture media. As mentioned elsewhere herein, fetal bovine serum is obtained from a bovine fetus, via a closed collection system such as at a slaughterhouse and is not intended to be manufactured in a sustainable or cost-effective way. In some embodiments, a similar product, e.g., bovine serum, can be obtained in a sustainable way, e.g., using regenerative farming practices described herein. For example, plasma may be withdrawn, for example, every 24 hours, from a single cow or from multiple different cows. Blood plasma comprises fibrinogen, which when stimulated to crosslink, e.g., by addition of thrombin, produces bovine serum that is rich in many of the same growth factors as fetal bovine serum. However, unlike fetal bovine serum, regenerative farming allows the serum to be repeatedly produced over the course of an animal's lifetime, thus reducing the cost of manufacture while simultaneously reducing the carbon footprint associated with caring for large animals. For example, it is estimated that a single cow may provide enough blood product over its lifespan, e.g., 15-20 years, to produce approximately 22,000 pounds of cultivated meat products (equal to 50 cattle at 440 pounds of beef per animal).

Some embodiments are directed toward methods of quantifying the decrease in the carbon footprint of the regenerative farm. For example, in some embodiments, the method comprises harvesting whole blood from a non-human animal, separating the whole blood into blood components, and using the blood components to produce a cell-based meat product.

Other embodiments are directed toward methods comprising raising a non-human living animal on a regenerative farm, withdrawing a plurality of blood draws from the non-human living animal, and forming a plurality of cultivated meat products using the plurality of blood draws. In other embodiments, the methods comprise raising a non-human living animal on a regenerative farm, harvesting at least one blood product and at least one other product from the non-human living animal, and using the at least one blood product to cultivate a cell-based meat. In some embodiments, the at least one other product comprises milk, wool, eggs, manure, etc. In some embodiments, the regenerative farm has a net zero carbon footprint.

In other embodiments, the method comprises grazing a non-human animal on a plurality of pastures such that the non-human animal increases the carbon content of each of the pastures by at least 50 mass % due to its waste, relative to identical pastures in the absence of the non-human animal, withdrawing a plurality of blood draws from the non-human animal, and using the blood draws to produce a cell-based meat product. Rotating the field that the non-human animals graze, also known as regenerative grazing, is a regenerative farming practice that uses the manure produced by grazing animals to fertilize and maintain soil health. In some embodiments, the non-human living animals comprise multiple different species, such as for example, cow, goat, sheep, horses, etc. In some embodiments, regenerative grazing comprises rotating the grazing of the non-human animals on a plurality of pastures. In some embodiments, the pastures may be rotated every day, every second day, every third day, every fourth day, every fifth day, every sixth day, every seventh day, every 2 weeks, every 4 weeks, every 8 weeks, every 16 weeks, every 32 weeks, or every 52 weeks. In some embodiments, rotational grazing may reduce the greenhouse gas emissions by between 10 mass % and 50 mass %, relative to identical pastures in the absence of rotational grazing.

Accordingly, some aspects of the present disclosure are directed toward methods of sustainably producing a tissue, such as a cultivated meat product, for food and other applications using, for example, the harvested blood components obtained from a blood draw, e.g., a first blood draw. For example, in one embodiment, one or more blood products may be used to produce a cell culture growth media. In some embodiments, the method comprises obtaining components of a cell-culture medium by harvesting blood components, e.g., platelet-rich plasma, from a living animal to produce a cell-culture medium suitable for in vitro cell culture and culturing cells in said cell-culture growth medium. In some embodiments, the cell-culture growth media comprises a cell-culture media. Exemplary cell-culture medias that may be purchased by commercial vendors (e.g., Gibco, Sartorius, etc.), or synthetized by those skilled in the art, include DMEM, RPMI 1640, MEM, DMEM/F12, Ham's F-10 nutrient mixture, Ham's F-12 nutrient mixture, Media 199, Plasma-Lyte, PBS, etc. In some instances, a sustainably harvested blood products, e.g., platelet-rich plasma and/or a platelet lysate, obtained from a first blood draw may be added to the cell-culture media, for example, to enhance the biological activity of the cell-culture media, e.g., to promote cell proliferation. In some embodiments, the concentration of platelet-rich plasma and/or platelet lysate is at least 2% by weight, at least 5% by weight, at least 10% by weight, at least 15% by weight, at least 20% by weight, etc. of the cell culture growth medium. In some embodiments, cultivating the tissue from a first blood draw comprises exposing cells in the first blood draw to the cell growth media.

As another example, the harvested blood products may be used to produce a microcarrier or other scaffold comprising fibrin. Non-limiting examples of these may be found in U.S. Pat. Apl. Ser. No. 63/279,617, filed Nov. 15, 2021, entitled “Constructs Comprising Fibrin or Other Blood Products for Meat Cultivation and Other Applications,” by Hosseini, et al., incorporated herein by reference in its entirety. Fibrin is an edible fibrous protein involved in the clotting of blood. It can be formed, for example, by the action of the protease inhibitor thrombin on fibrinogen, which causes it to polymerize and form a clot. Fibrin can be used as a passive scaffolding material in some embodiments. However, in some embodiments, fibrin can specifically bind certain growth factors in the cell culture media that promote cell adhesion, proliferation, and migration. Non-limiting examples include fibronectin, hyaluronic acid, von Willebrand factor, or the like.

In certain embodiments, microcarriers or scaffolds such as those discussed herein may be treated to facilitate binding of cells, such as myoblasts. Additional non-limiting examples of microcarriers or scaffolds may be found in U.S. Pat. Apl. Ser. No. 63/159,403, filed Mar. 10, 2021, entitled “Constructs for Meat Cultivation and Other Applications,” by Khademhosseini, et al. For example, the microcarriers or scaffolds may be exposed to a non-human serum, which may include growth factors that bind to the microcarriers or scaffolds. The growth factors may, for example, promote cell adhesion, proliferation, and/or migration of cells into the microcarriers or scaffolds.

In some embodiments, the microcarriers or scaffolds may comprise any material that forms an edible hydrogel, such as fibrin. For example, in one embodiment, a microcarrier may be formed from a non-human blood plasma, or platelet rich plasma (PRP), both of which contain plasma-rich fibrinogen that can be crosslinked or otherwise processed to form a fibrin hydrogel. Such crosslinking can be achieved by exposure to thrombin, calcium, or other conditions such as those described herein. In some embodiments, fibrin hydrogels are formed using non-human blood plasma, and/or PRP, containing fibrinogen, e.g., at least 10 wt %, or more in some cases.

In certain embodiments, non-human cells such as myoblasts and adipose cells may be seeded on the microcarriers or other scaffolds and grown in a bioreactor to produce a cultivated meat product. For instance, myoblasts may be grown on microcarriers and, in some embodiments, allowed to differentiate or fuse to form aligned myotubes, e.g., within a bioreactor or other cell culture system.

As another example, the harvested blood products may be used to produce fatty-like tissues. An emulsion of fat may be prepared, for example, by emulsifying fat with non-human blood plasma. In some cases, the fat may be caused to form a fat emulsion by mixing the fat with non-human blood plasma. Without wishing to be bound by any theory, it is believed that the plasma has components that can emulsify fat to form fat particles such as chylomicrons. For instance, the plasma may include proteins or surfactants that can from such fat particles. The non-human blood plasma may be treated in some embodiments to form a fat replica. For example, fibrin within the plasma may be caused to clot and/or by causing the fibrin to crosslink, e.g., by exposing it to thrombin, calcium, or other clotting agents such as those described herein. In addition, in some embodiments, a fat replica may comprise a fat emulsion contained within a hydrogel. The hydrogel may be formed from non-human blood plasma, e.g., as discussed, and/or another component. Non-limiting examples of such hydrogels include alginate, gelatin, or others such as those described herein. See, for example, U.S. Pat. Apl. Ser. No. 63/279,642, filed Nov. 15, 2021, entitled “Systems and Methods of Producing Fat Tissue for Cell-Based Meat Products,” by Hosseini, et al., incorporated herein by reference in its entirety.

In other embodiments, the harvested red blood cells may be used to provide heme or heme-containing proteins. For instance, in one embodiment, the heme may be obtained from non-human red blood cells, e.g., cultivated within the bioreactor. For example, red blood cells may be lysed, e.g., by exposing the cells to hypoosmotic or distilled water to form a lysate of non-human red blood cells. Red blood cells contain hemoglobin, a structurally similar protein to myoglobin. Hemoglobin also contains a heme moiety. Lysing the red blood cells may release hemoglobin, e.g., into solution. Furthermore, in addition to these, other methods of lysing red blood cells can be used. See, for example, U.S. Pat. Apl. Ser. No. 63/279,644, filed Nov. 15, 2021, entitled “Production of Heme for Cell-Based Meat Products,” by Hosseini, et al., incorporated herein by reference in its entirety.

Some aspects of the disclosure are related to forming a cultivated meat product grown, for example, using the harvested blood components from a blood draw as described herein. In some embodiments, the cultivated meat product comprises a muscle replica (e.g., muscle cells optionally grown on a microcarrier, e.g., a fibrin microcarrier), a fatty-like tissue, and/or a lysate of red blood cells. In some embodiments, the cultivated meat product comprises. The muscle replica, fatty-like tissue, and lysate of red blood cell may be present in any suitable amount with the cell-based meat product. For example, the muscle replica, fatty-like tissue, and lysate of red blood cell may be present in at least 10 wt %, at least 20 wt %, at least 30 wt %, at least 40 wt %, at least 50 wt %, at least 60 wt %, at least 70 wt %, at least 80 wt %, at least 90 wt %, etc., and/or no more than 95 wt %, no more than 90 wt %, no more than 80 wt %, no more than 70 wt %, no more than 60 wt %, no more than 50 wt %, no more than 40 wt %, no more than 30 wt %, no more than 20 wt %, no more than 10 wt %, etc., of the cell-based meat product.

Further, in some embodiments, structures resembling whole cuts of meat can be produced by binding together fatty-line and meat-like tissues by various chemical and enzymatic reactions known to the art such as transglutaminase or meat glue to make the whole cut of meat, although such steps may not be necessary for the production of ground meat.

In another set of embodiments, provided herein are certain aspects that provide a comprehensive sustainable method of growing cells in a cell culture cell, e.g., a bioreactor. Some embodiments, for example, comprise the steps of obtaining components of a cell-culture medium by sustainably performing blood draws from living non-human animals to produce a cell-culture medium, comprising blood components, suitable for in vitro cell culture and culturing cells in said cell-culture medium. The method is useful, for example, for producing cell-based cultivated meat by cell culturing, e.g., because of the massive scale required to do so on a global scale. Cell-based cultivated meat is a recent innovation in the food industry and is manufactured using animal cells in vitro condition and in culture medium to create a meat without farming animal in traditional way. As used herein, cell-based meat is synonymous with cultivated meat, cultured meat, cellular meat, slaughter-free meat, and synthetic meat, among other related terms.

Culture media for production of cell-based meat includes, but is not limited to, proteins, peptides, vitamins, cytokines and growth factors extracted from animal blood. Such compositions may be extracted in certain embodiments from a blood draw performed on a living animal, where the animals are raised or “farmed” for the purpose of sustainably extracting blood, milk, wool and other resources. For example, animals that are farmed for harvesting milk can also be used for harvesting of both blood and milk. For instance, harvesting may occur without killing the animals.

By “sustainably harvesting blood components,” it is intended that blood components can be extracted from a blood draw in a manner which does not kill the non-human animal. In some embodiments, this includes any method by which blood and/or blood products can be removed from an animal in a manner that permits selected components, e.g., nutrients, some portion of red blood cells, platelets, fibrinogen, plasma or serum including growth factors to be harvested and other blood components, for example, red blood cells, to be returned to the animal without killing the animal. Such methods include, for example, apheresis, plasmapheresis, erythrocytapheresis, plateletpheresis, leukapheresis, centrifugation and the like. As used herein, “apheresis” refers to any process by which blood or blood products are removed from an animal and one or more components are separated from the blood or blood product with the remaining components returned to the animal's circulation. In some embodiments, performing apheresis returns at least 50 vol %, at least 60 vol %, at least 70 vol %, at least 80 vol %, or at least 90 vol % of a first blood draw back to the non-human living animal. In some embodiments, performing apheresis returns at least 50 vol %, at least 60 vol %, at least 70 vol %, at least 80 vol %, or at least 90 vol % of a second blood draw back to the non-human living animal. In some embodiments, the first blood draw occurs before the second blood draw. In other embodiments, the first blood draw may occur after the second blood draw. There may also be more than two blood draws. For example, there may be at least 3, at least 4, at least 5, at least 6, at least 8, at least 10, at least 15, at least 20, at least 25, at least 30, at least 40, at least 50, at least 60, at least 70, at least 80, at least 90, at least 100, etc. blood draws from a particular animal.

In some embodiments, platelets may be separated from a first blood draw prior to returning the first blood draw to the non-human living animal. In some embodiments, the platelets are separated from the first blood draw using apheresis. In other embodiments, a platelet-rich plasma is separated from the first blood draw prior to returning the first blood draw to the non-human living animal. In some embodiments, separating the platelet-rich plasma from the first blood draw uses apheresis. In some embodiments, platelets may be separated from a second blood draw prior to returning the second blood draw to the non-human living animal. In some embodiments, the platelets are separated from the second blood draw using apheresis. In other embodiments, a platelet-rich plasma is separated from the second blood draw prior to returning the second blood draw to the non-human living animal. In some embodiments, separating the platelet-rich plasma from the second blood draw uses apheresis.

As will be appreciated by those of skill in the art, apheresis may increase the concentration of the extracted blood component. For example, in some embodiments, a platelet-rich plasma separated from a first blood draw using apheresis comprises at least 2 times the normal concentration of platelets in the first blood draw. In some embodiments, the concentration of the platelet-rich plasma extracted from the first blood draw using apheresis is at least 10⁵ platelets/mL. For example, in some embodiments, a platelet-rich plasma separated from a second blood draw using apheresis comprises at least 2 times the normal concentration of platelets in the second blood draw. In some embodiments, the concentration of the platelet-rich plasma extracted from the second blood draw using apheresis is at least 10⁵ platelets/mL.

While some methods are directed to the sustainable production of components for cell-culture media, blood and blood products collected from slaughterhouses, e.g., for production of traditional meat products, can be used to supplement the production of cell-based meat. For example, apheresis machines can be used in some embodiments to separate blood components, e.g., the platelets, plasma or platelet rich plasma, from an animal's blood and return used to return other cells, e.g., red blood cells, back to the animal's body. Plasma and platelets can be donated more frequently than red blood cells since their turnover is faster in animal bodies.

In some embodiments, the blood components can be extracted into blood bag, e.g., comprising one or more anticoagulants, and processed, e.g., at a later time, for example, by centrifugation at 300 g (range 100-500 g) for at least 25 min (range 10-50 min). Other techniques are also possible in other embodiments, e.g., as described herein. In some embodiments, the platelets, plasma or platelet rich plasma may be harvested from the animal, while red blood cells are returned to the animal.

According to one embodiment, platelet rich plasma (PRP) may be obtained from a non-human living animal and used for cell culturing. In some embodiments, cell culture media comprising platelet rich plasma alone has been found to be more effective on cell proliferation, compared to platelet concentrate or plasma alone. This may be particularly useful in certain embodiments because the separation process of PRP may be faster under certain conditions than the isolation of platelet concentrate or plasma alone, and/or can be performed in single step. In some embodiments, PRP may be obtained from any species with blood such as cows and sheep. See also U.S. Ser. No. 63/164,397 filed Mar. 22, 2021, the disclosure of which is hereby incorporated by reference.

In some embodiments, the separated platelets and/or platelet-rich plasma can be lysed by using, for example, a freeze-thawing process, physical shearing such as sonication to release their content including cytokines and growth factors, or other suitable techniques. In some embodiments, the separated platelets and/or platelet-rich plasma can be treated chemically and/or biochemically, such as by addition of calcium ion, thrombin or other platelet activating reagents such as citrate, EDTA, calcium chloride, plasminogen to release cytokines and growth factors.

According to another aspect, a portion of red blood cells obtained during apheresis, and/or as a byproduct during platelet-rich plasma (PRP) donation, can be lysed to release heme and used for enhancement of color and taste of cell-based meat products.

In some embodiments, the cell culture growth media may comprise PRP as a supplemental growth media. In some embodiments, the PRP may be further supplemented with additional blood derived proteins, peptides, vitamins, cytokines, and/or growth factors, etc. In certain embodiments, components such as synthetic and/or recombinant proteins, peptides, vitamins, cytokines, growth factors, etc., e.g., similar to those in plasma, can be added to the growth media, for example, to boost the proliferation or differentiation of cells to cell line such adipocyte or myofibers.

Various blood components, e.g., PRP, may be added to a basal culturing media for the production of a cell-based meat product. In some cases, the media may be used to enhance the proliferation of primary cells such as myoblasts, fibroblasts, adipocyte, vascular, epithelial cells, mammalian glands, osteoblasts, tenocyte, neural cells, etc., e.g., which in some embodiments may be isolated from vertebrate and invertebrate animals, e.g., that are used as source of edible proteins worldwide. These may include but are not limited to non-human animals such as cows, sheep, swine, horses, goats, camels, whales, fishes, crabs, shrimps, bison, crocodile, and the like.

In some embodiments, cell culture growth media comprising blood components may be used to cultivate a cell-based meat, e.g., that is made from stem cells such mesenchymal stem cells, induced pluripotent stem cells, and the like, e.g., to originate different cell types necessary for meat development.

In some embodiments, the cell culture growth media comprising PRP extracted from blood of one species can be used for the cultivation of cells from other species or for cultivation of cells from the same species from which it was obtained.

In some embodiments, the cell culture growth media comprising PRP can be used to cultivate a texturized meat fiber that resembles real meat. In some embodiments, fibrin can be used as a cell scaffold or cell microcarrier to cultivate cultivation of cells in bioreactors.

In some embodiments, PRP can be used in cell culture to provide a meat-like texture to the cultured cells, e.g., by allowing the PRP to form fibrin hydrogels by neutralizing an anticoagulant. For instance, addition of calcium ion and thrombin or activator of prothrombin allows the initiation of coagulation cascade of soluble fibrinogen in PRP. Subsequently, the fibrin gel can be physically treated by different mechanical approaches such as pressing, agitation, shearing, milling to induce a fibrillar meat-like texture. Such meat-like texture can be used as a biosimilar to ground beef or may subsequently bind together with protein cross-linkers such as glutaminase to improve mechanical characterization that resemble a cut of meat. Harvested red blood cells (RBCs) and their lysates can be incorporated into the meat products for their gustatory, olfactory and visual attributes. Other examples using fibrin hydrogels may be seen in U.S. Pat. Apl. Ser. No. 63/279,617, filed Nov. 15, 2021, entitled “Constructs Comprising Fibrin or Other Blood Products for Meat Cultivation and Other Applications,” by Hosseini, et al., incorporated herein by reference in its entirety.

In some embodiments, PRP at a concentration of 10% (ranging from 1-100%, 5-50%, 5-20%, or the like) can be added to a culturing media or a solution containing Ca²⁺ ions or other aiding agent such as thrombin to promote coagulation.

In some embodiments, prior to formation of a fibrin gel, the cells that are grown such as discussed herein can be mixed with PRP in a desired concentration at a cell density, for example from 10 million to 100 million cells/ml, and/or ranging from 20% to 90% w/w to fibrin (e.g., 80% w/w to fibrin) and can be processed as discussed herein.

If the color and taste of red meat is desirable, a red blood cell lysate obtained from animals may in some embodiments be added at a concentration of 2% (e.g., ranging 0.5% to 8%) to plasma or PRP at concentration of 10% (e.g., ranging from 5% to 95% in coagulating solution e.g., DMEM or CaCl2)) and processed according above method to obtain a red meat-like structure with improved color and taste. See also U.S. Pat. Apl. Ser. No. 63/279,644, filed Nov. 15, 2021, entitled “Production of Heme for Cell-Based Meat Products,” by Hosseini, et al., incorporated herein by reference in its entirety.

According to another aspect, PRP can be added to an adipocyte cell culture as above and allowed to clot and processed as described herein, e.g., to obtain a whitish fatty-like tissue. For example, it is possible in certain embodiments to mix plant-based fats such as coconut oil or milk fat (butter oil) with PRP to obtain fat-like fibrin tissue. See, for example, U.S. Pat. Apl. Ser. No. 63/279,642, filed Nov. 15, 2021, entitled “Systems and Methods of Producing Fat Tissue for Cell-Based Meat Products,” by Hosseini, et al., incorporated herein by reference in its entirety.

In some embodiments, fatty-like tissue and meat-like tissue obtained as described herein can be mixed together, e.g., by different methods, including extruding using a 3D printer to form anisotropic structure with meat-like texture.

Further, structures resembling whole cuts of meat can be produced in certain embodiments by binding together fatty-like and meat-like tissues, e.g., by using various chemical and enzymatic reactions known to those of ordinary skill in the art such as transglutaminase or meat glue to make the whole cut of meat, although such steps may not be necessary in other embodiments for the production of ground meat.

A sustainable and retainable replacement for FBS is needed in certain embodiments, e.g., to bring cell-based meats to the market. In one set of embodiments, human platelet lysate (PL) can be used as a replacement for FBS for cultivating meat products. Human PL can be used as a growth supplement on many types of cells, such as stem cells, according to some embodiments. Human PL may be obtained as a byproduct of platelet donation for other therapeutic proposes. Usually, the donated platelets have an expiration date of a week, e.g., after collection, and after that it may be frozen, and with thawing it is called a human PL. Since the platelets may have a variety of growth factors, after thawing, they are lysed to release growth factors, which may be useful for growing cells. In fact, this is a native role of platelets, which release growth factors at a site of vascular injury to promote healing of injured tissues. Despite several application of human platelet lysate for therapeutic applications such as wound healing, or to aid the healing other injuries such as tendons and cartilage and culture of human cells for therapeutic application, animal platelet lysate has not been considered for therapeutic proposes. However, it should be understood that the present disclosure is not limited to only human platelet lysate, and in other embodiments, non-human platelet lysate can be used, e.g., in addition to or instead of human platelet lysate.

For example, animal derived PL or Platelet Rich Plasma (PRP) from different animals such as bovine, equine and canine have also been used for the treatment of a wide range of injuries in veterinary medicine. PL and/or PRP can be donated by a non-human animal, e.g., without causing health issues. Accordingly, in certain embodiments, PL and/or PRP may be used as is discussed herein.

For example, substitution of bovine PL and PRP produced by sustainable extraction from cows and/or other farm animals, e.g., as discussed herein, for example, to obtain growth factors for cell culture growth media could, in some embodiments, substantially reduce the cost of such media and/or of cultivated meat produced therefrom, e.g., to levels competitive with that of meats produced from slaughtered livestock. Not only might it be the case that the mass efficiency of cell-based meat production within the period of 16 months which is used to grow farm cattle in farm for slaughter is 5×greater, but a cow may be able to produce components for cell culture media sufficient to produce meat equal to 50 cattle during its 15-20 years life span. This means that all environmental impacts of animal-based meat can be reduced by, e.g., 98% by using farms for PL or PRP production for cell-based meat factories. Since such animal growth factors may be important components for cell proliferation, it is possible according to certain embodiments to add one or more growth factors and/or other components such as plasma, e.g., that carries a number of nutritional factors to boost its efficacy.

As will be understood by those in the art, the above discussions are not limited to the production of cultivated meat products. For example, in some embodiments, the methods described herein may be used to produce other cultivated products in a sustainable way from a non-human living animal. In other embodiments, the method comprises withdrawing a plurality of blood draws from a non-human living animal without killing the animal and forming a plurality of cultivated products using the plurality of blood draws. The cultivated product may be any product capable of being grown in a bioreactor, such as skin, milk, wool, organs, horn, tusks, leather, milk, hair, fur, and the like, including any of those previously discussed herein.

The following are each incorporated herein by reference in their entireties: U.S. Provisional Patent Application Ser. No. 63/159,403, filed Mar. 10, 2021, entitled “Constructs for Meat Cultivation and Other Applications”; U.S. Provisional Patent Application Ser. No. 63/279,617, filed Nov. 15, 2021, entitled “Constructs Comprising Fibrin or Other Blood Products for Meat Cultivation and Other Applications”; U.S. Provisional Patent Application Ser. No. 63/279,631, filed Nov. 15, 2021, entitled, “Methods and Systems of Preparing Cultivated Meat from Blood or Cellular Biomass”; U.S. Provisional Patent Application Ser. No. 63/279,642, filed Nov. 15, 2021, entitled, “Systems and Methods of Producing Fat Tissue for Cell-Based Meat Products”; U.S. Provisional Patent Application Ser. No. 63/279,644, filed Nov. 15, 2021, entitled “Production of Heme for Cell-Based Meat Products”; US Provisional Patent Application Serial No. U.S. 63/300,577, filed Jan. 18, 2022, entitled “Animal-Derived Antimicrobial Systems and Methods”; U.S. Provisional Patent Application Ser. No. 63/164,397, filed Mar. 22, 2021, entitled “Growth Factor for Laboratory Grown Meat”; U.S. Provisional Patent Application Ser. No. 63/164,387, filed Mar. 22, 2021, entitled, “Methods of Producing Animal Derived Products”; U.S. Provisional Patent Application Ser. No. 63/314,171, filed Feb. 25, 2022, entitled “Growth Factors for Laboratory Grown Meat and Other Applications”; and U.S. Provisional Patent Application Ser. No. 63/314,191, filed Feb. 25, 2022, entitled “Methods and Systems of Producing Products Such as Animal Derived Products.”

Numerous modifications and variations in the practice of the present disclosure are expected to occur to those skilled in the art upon consideration of the foregoing description on the presently preferred embodiments thereof. Consequently, the only limitations which should be placed upon the scope of the present invention are those that appear in the appended claims.

The following examples are intended to illustrate certain embodiments of the present disclosure, but do not exemplify the full scope of the disclosure.

### Example 1

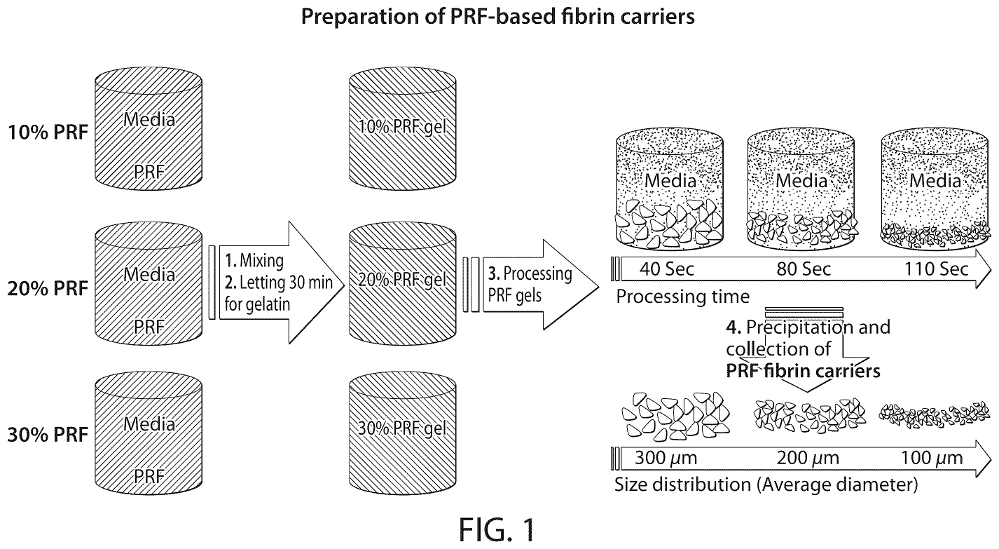

This example demonstrates the performance of platelet-rich plasma isolates from different cows. Table 1 contains information related to the types of cows used, their date of birth, weight, total volume of donated platelet-rich plasma, and donation frequency. The platelet-rich plasma from each animal was then taken and used to produce a cell-culture growth medium. To determine the proliferative potential of such solutions, bovine myoblasts were suspended in DMEM containing 10% fetal bovine serum and seeded at a cell density of 10,000 cells/well to allow for adhesion to the culture substrate. After 6 hours, the culture media was removed and fresh DMEM (serum free) was added. After 12 hours, the cell culture media was again removed and replaced with DMEM containing 10% bovine platelet-rich plasma isolates from the various cows. Control samples were incubated with 10% fetal bovine serum or 10% porcine platelet-rich plasma (obtained from a slaughterhouse). The cells were subsequently incubated for 24 hours at 37° C. in 95% 02 and 5% CO₂, after which, the cell culture medium was removed. The cultured cells were then washed 3× with phosphate buffered saline to remove all media, trypsinized to release them from the culture substrate, and the cell numbers determined using optical techniques. The results indicated that the platelet-rich plasma, isolated from the bovine donors, significantly enhanced cell proliferation, relative to serum free media, and was equally effective as fetal bovine serum, regardless of the donor cow (FIG. 1). In addition, Table 1 shows information related to the types of cows used, their date of birth, weight, volume of donated platelet-rich plasma, and donation frequency.

TABLE 1
Young Holstein Hefer

| | DOB | Weight (lb) | Donated PRP (L) |
|---|---|---|---|
| 4266 | Jun. 18, 2019 | 1350 | 9.8 |
| 4321 | Jul. 19, 2019 | 1450 | 10.5 |
| 4348 | Jul. 31, 2019 | 1450 | 10.5 |
| Donation frequency: | | | |
| Weekly | | | |

Mature Holstein Cow

| Number | DOB | Weight (lb) | PRP (L) |
|---|---|---|---|
| 2317 | Jul. 17, 2016 | 1950 | 14.2 |
| 2348 | | 1950 | 14.2 |
| 2315 | | 1950 | 13.8 |
| Donation frequency: | | | |
| Biweekly | | | |

Young Holstein Hefer

| Number | DOB | Weight (lb) | PRP (L) |
|---|---|---|---|
| 14347 | Jul. 24, 2019 | 1250 | 9.1 |
| 14424 | Aug. 10, 2019 | 1250 | 9.1 |
| 14583 | Sep. 26, 2019 | 1250 | 9.1 |
| Donation frequency: | | | |
| Biweekly | | | |

Young Steer

| Number | DOB | Weight (lb) | PRP (L) |
|---|---|---|---|
| 5760 | Sep. 27, 2019 | 1400 | 10.2 |
| 5726 | Sep. 22, 2019 | 1350 | 9.8 |
| 5211 | May 26, 2019 | 1330 | 9.7 |
| Donation frequency: | | | |
| Biweekly | | | |

### Example 2

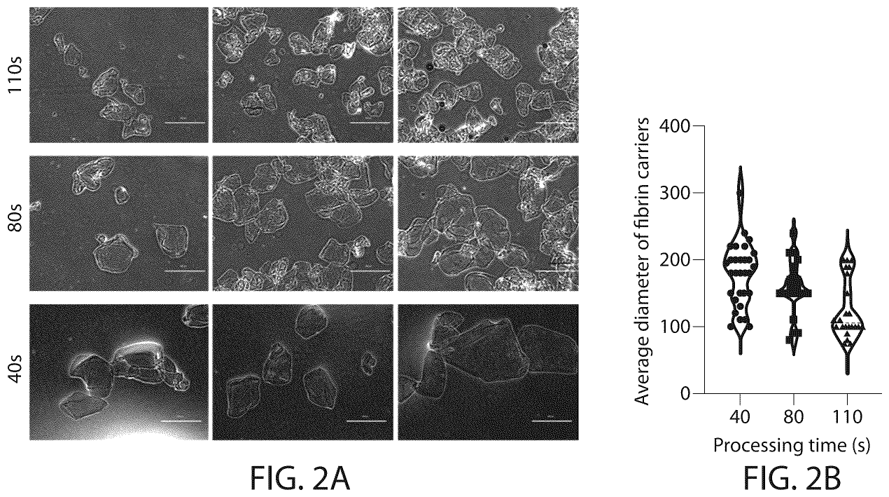

This example demonstrates the effect of varying the platelet-rich plasma concentration, isolated from various donor cows (see Table 1) on bovine myoblast proliferation. Platelet-rich plasma from a mature Holstein Cow (2371) and several young Holstein Heifers (4321, 4266, 4348, and 14583) was obtained and added to DMEM at a final concentration of 2.5 wt %, 5 wt %, or 10 wt %. To determine the proliferative potential of such solutions, bovine myoblasts were suspended in DMEM containing 10% fetal bovine serum and seeded at a cell density of 10,000 cells/well to allow for adhesion to the culture substrate. After 6 hours, the culture media was removed and fresh DMEM (serum free) was added. After 12 hours, the cell culture media was again removed and replaced with DMEM containing the varying concentrations of bovine platelet-rich plasma (as described above). Control samples were incubated in serum free DMEM or DMEM containing 10% fetal bovine serum. The cells were subsequently incubated for 24 hours at 37° C. in 95% 02 and 5% CO₂, after which, the cell culture medium was removed. The cultured cells were then washed 3× with phosphate buffered saline to remove all media, trypsinized to release them from the culture substrate, and the cell numbers determined using optical techniques. The results showed that cell proliferation increased with increasing concentrations of platelet-rich plasma; and that 10% platelet-rich plasma was as effective as 10% fetal bovine serum at promoting cell proliferation (FIGS. 2A-E).

### Example 3

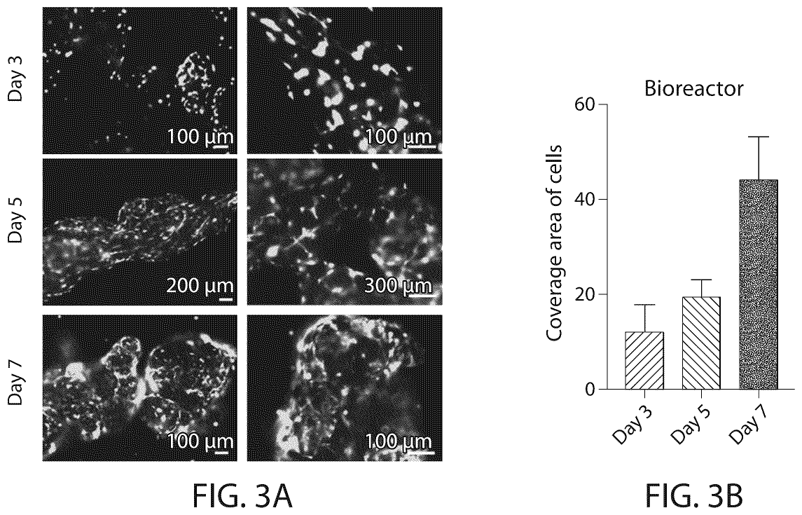

This example demonstrates the effect of varying the platelet-rich plasma concentration, isolated from various donor cows (see Table 1) on human hepatocyte proliferation. Platelet-rich plasma from a mature Holstein Cow (2371) and several young Holstein Heifers (4321, 4266, 4348, and 14583) was obtained and added to DMEM at a final concentration of 2.5 wt %, 5 wt %, 7.5 wt % or 10 wt %. To determine the proliferative potential of such solutions, human hepatocytes were suspended in DMEM containing 10% fetal bovine serum and seeded at a cell density of 10,000 cells/well to allow for adhesion to the culture substrate. After 6 hours, the culture media was removed and fresh DMEM (serum free) was added. After 12 hours, the cell culture media was again removed and replaced with DMEM containing the varying concentrations of bovine platelet-rich plasma (as described above). Control samples were incubated in serum free DMEM. The cells were subsequently incubated for 24 hours at 37° C. in 95% O₂ and 5% CO₂, after which, the cell culture medium was removed. The cultured cells were then washed 3× with phosphate buffered saline to remove all media, trypsinized to release them from the culture substrate, and the cell numbers determined using optical techniques. The results showed that cell proliferation increased with increasing concentrations of platelet-rich plasma with 10% platelet-rich plasma being the most effective (FIGS. 3A-F).

### Example 4

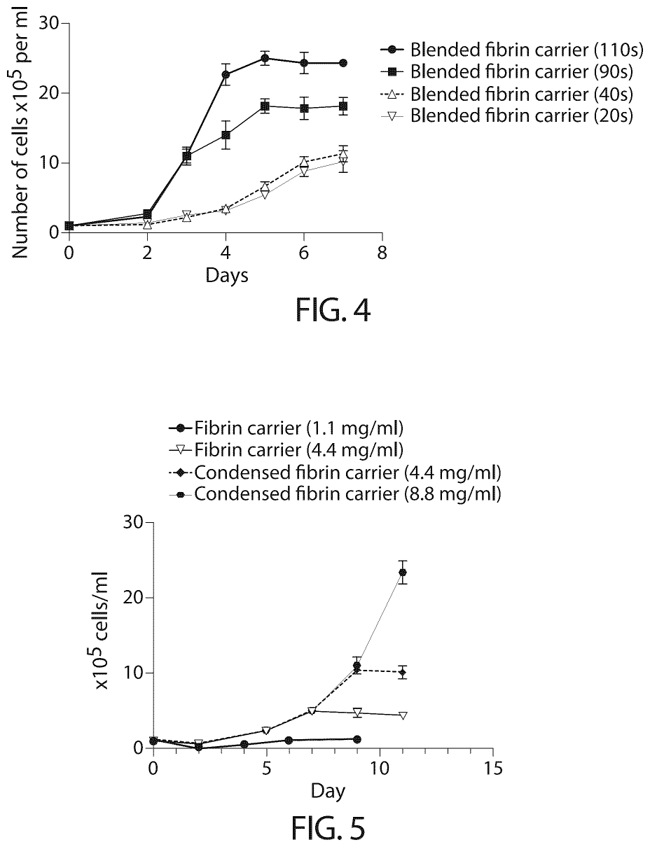

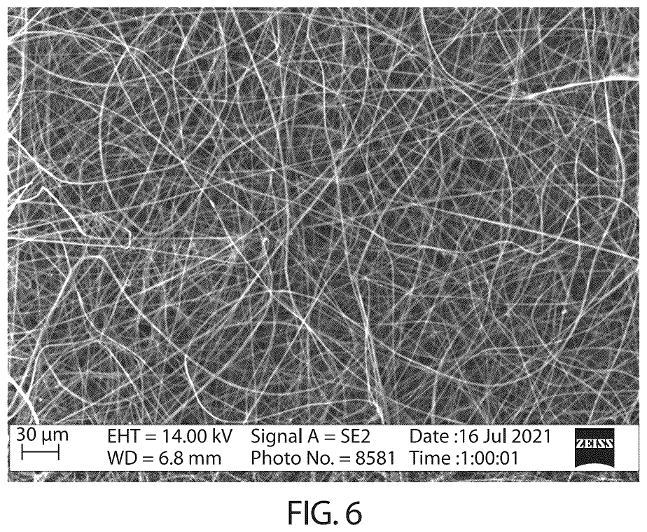

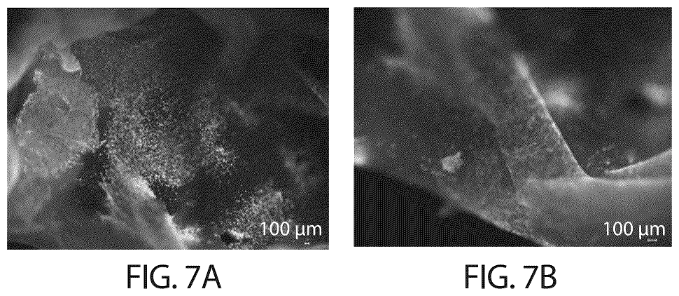

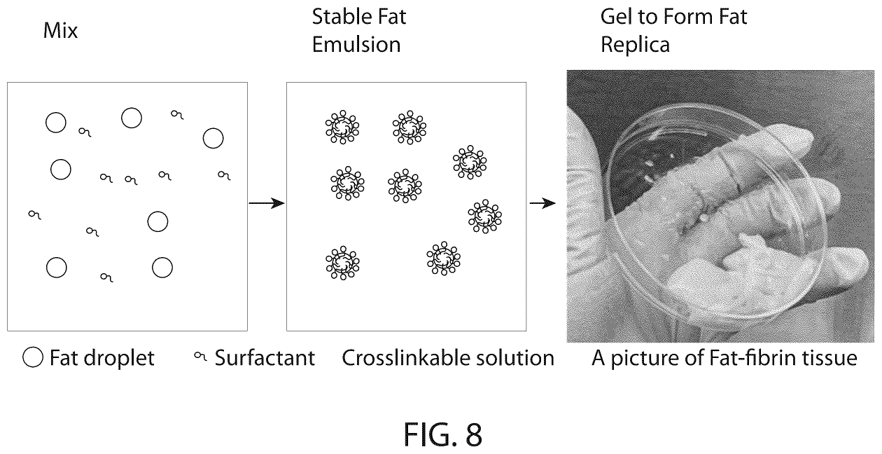

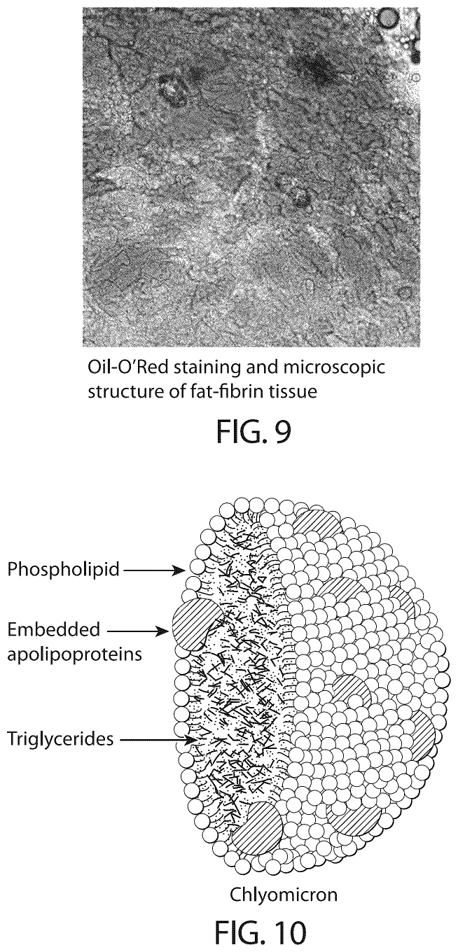

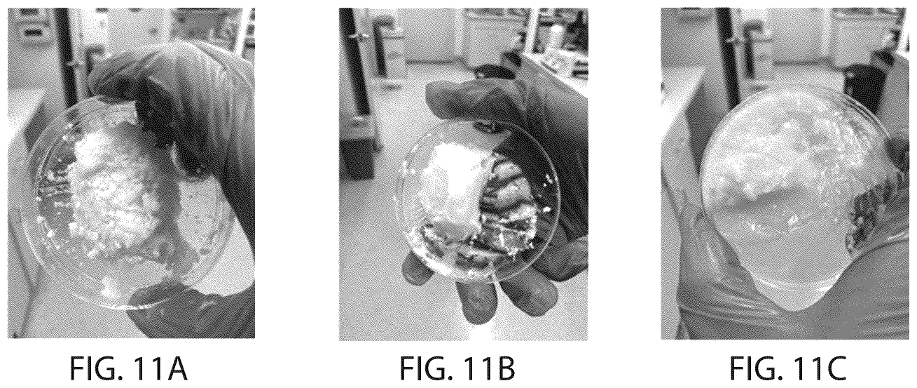

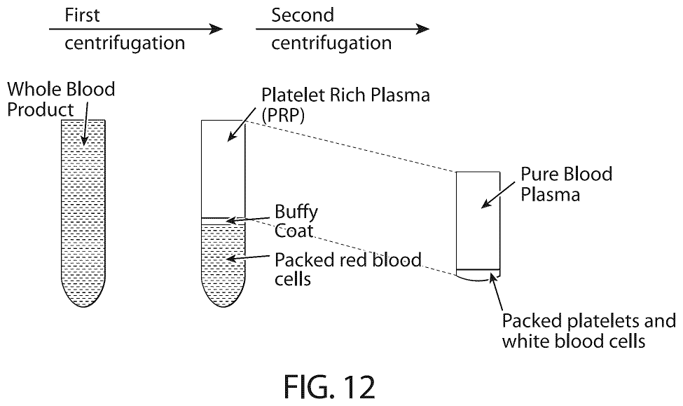

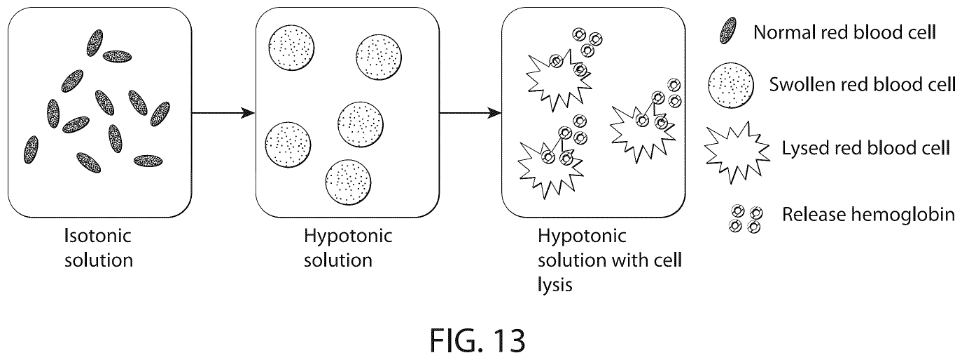

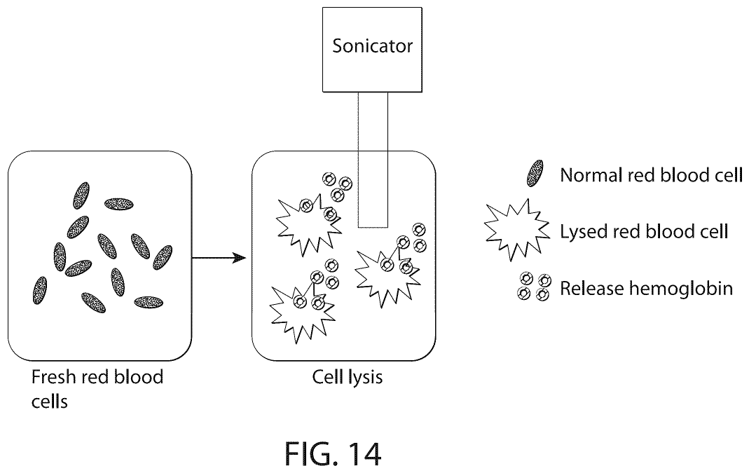

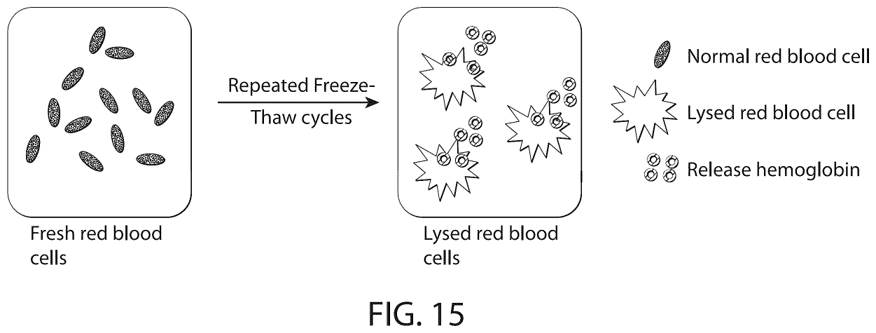

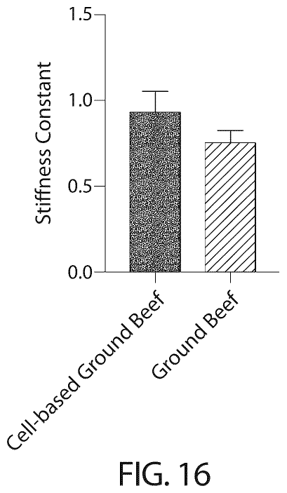

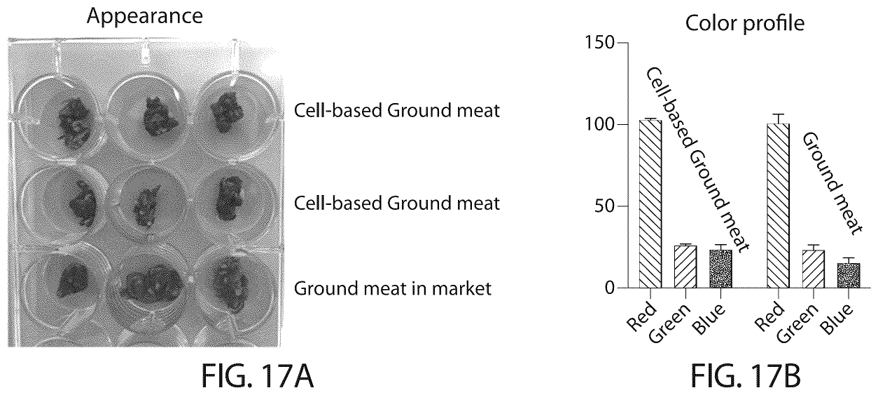

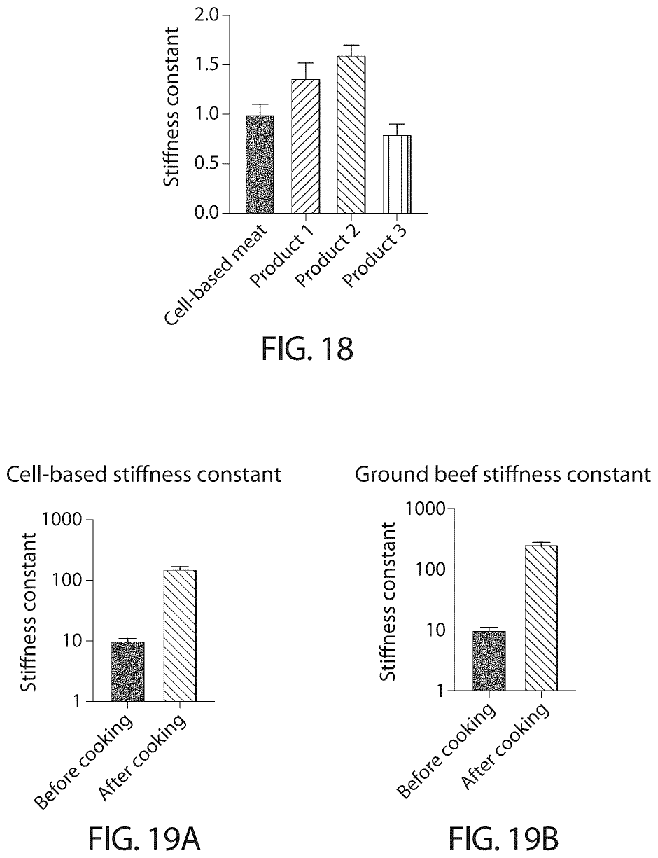

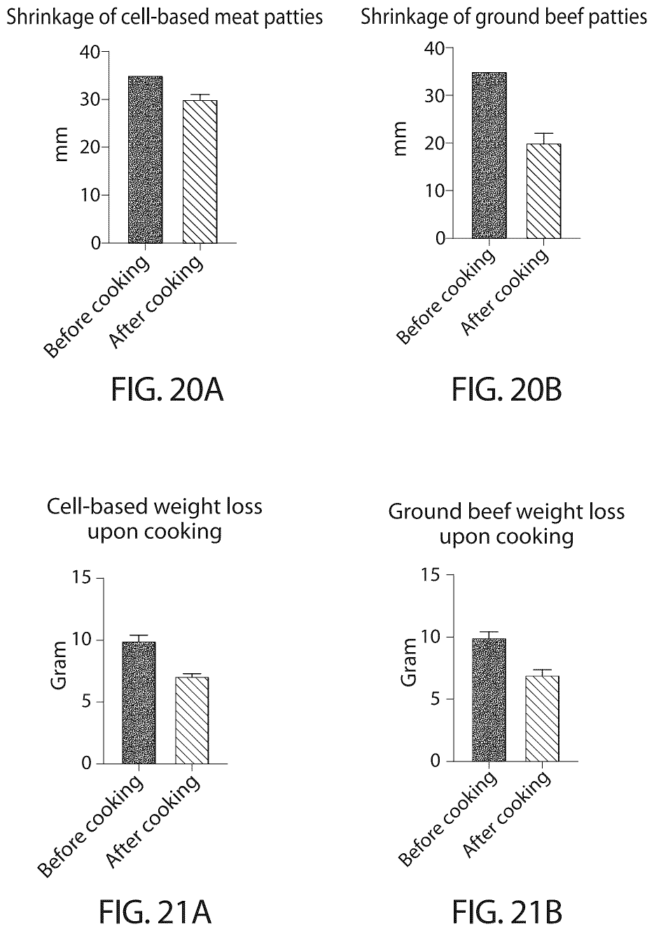

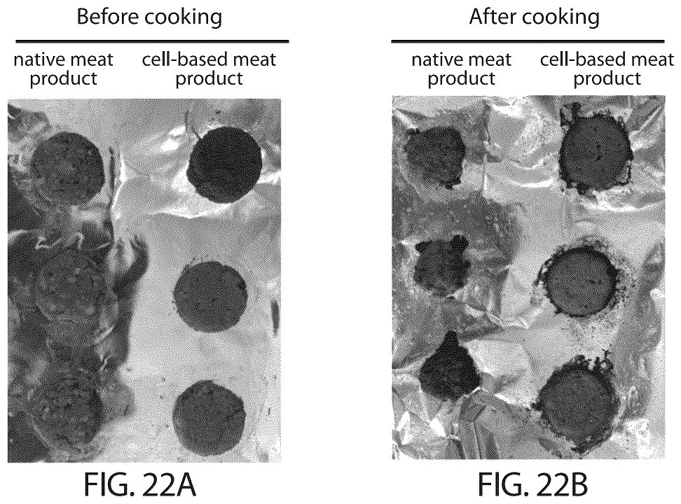

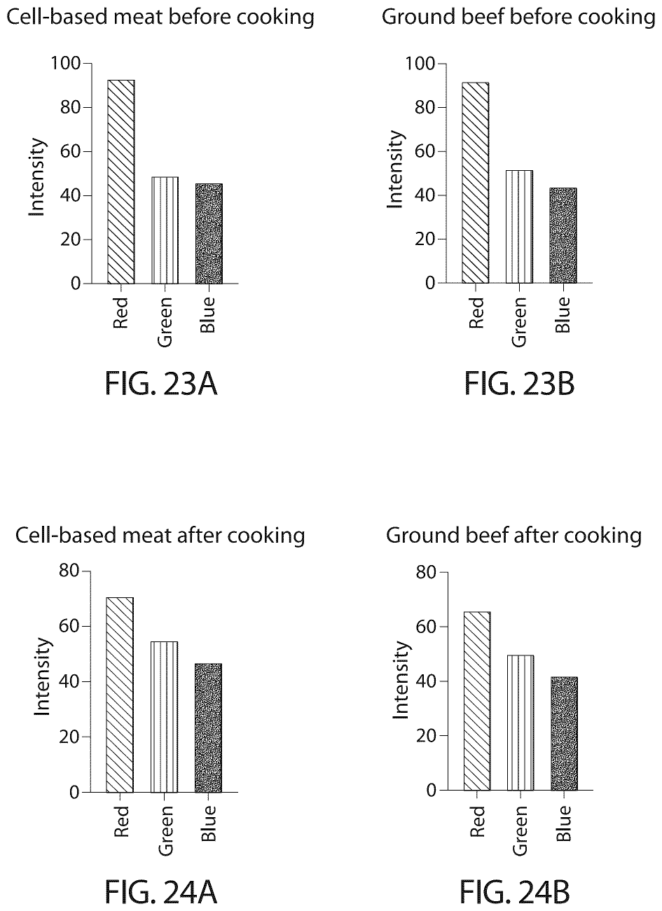

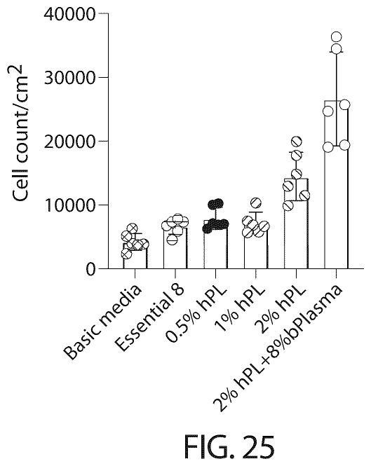

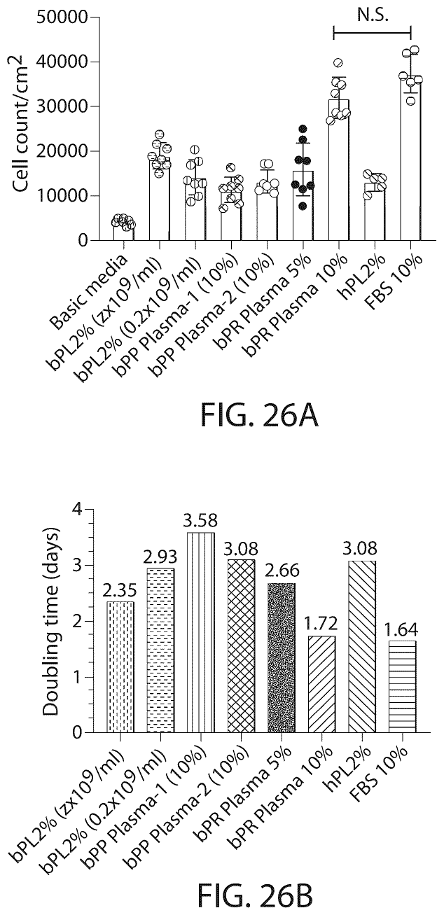

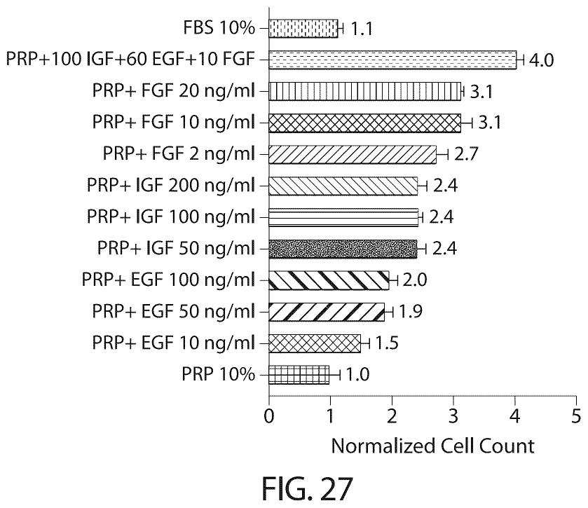

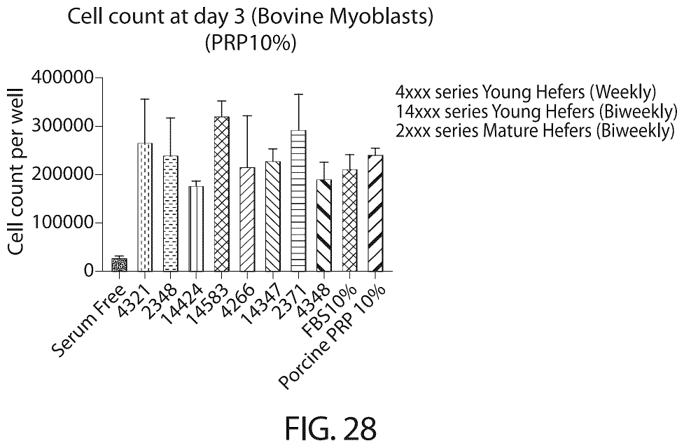

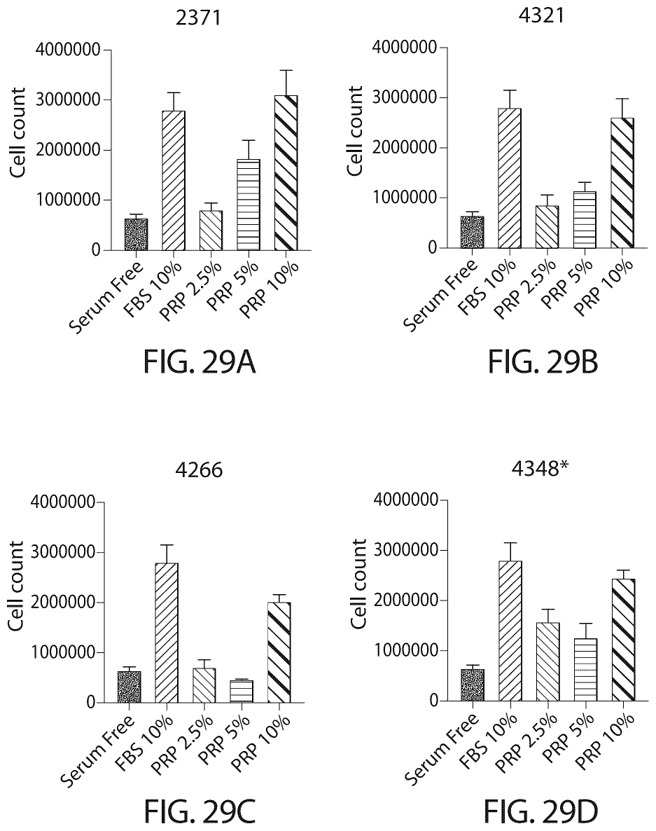

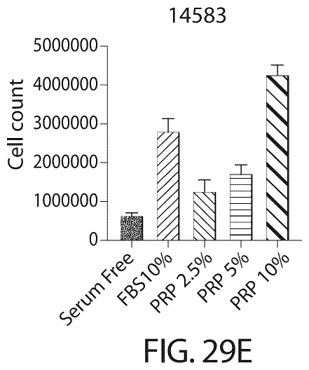

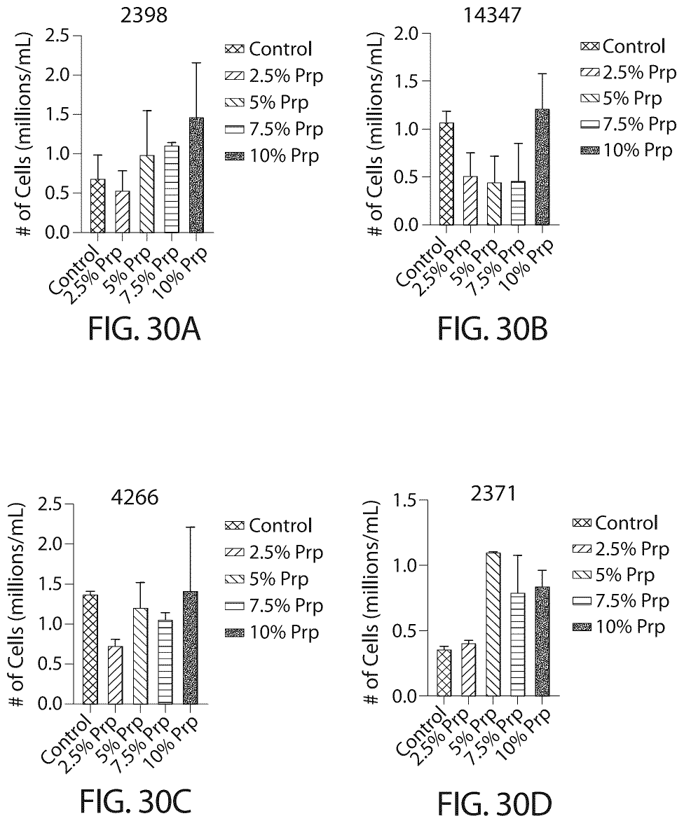

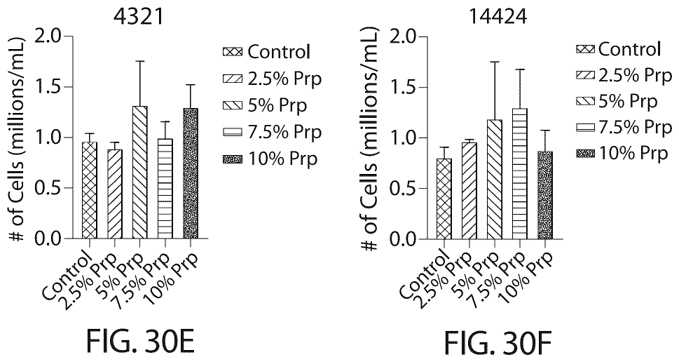

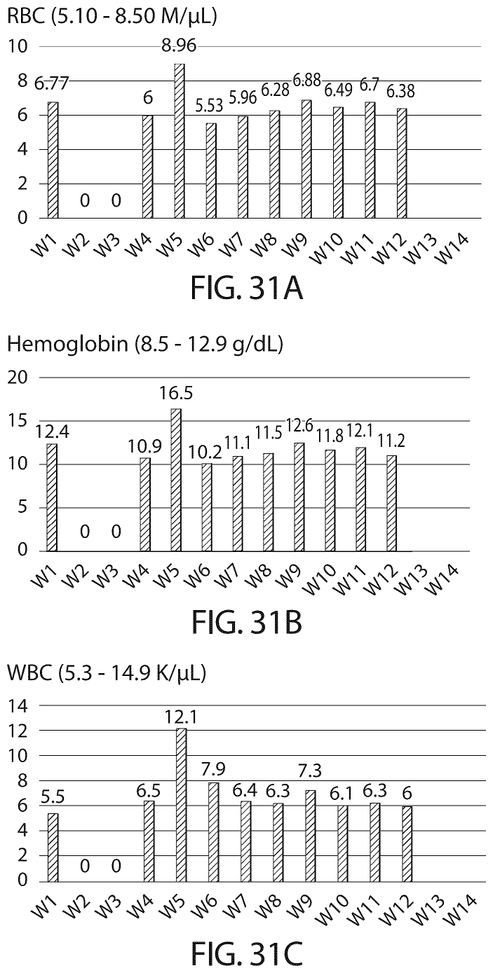

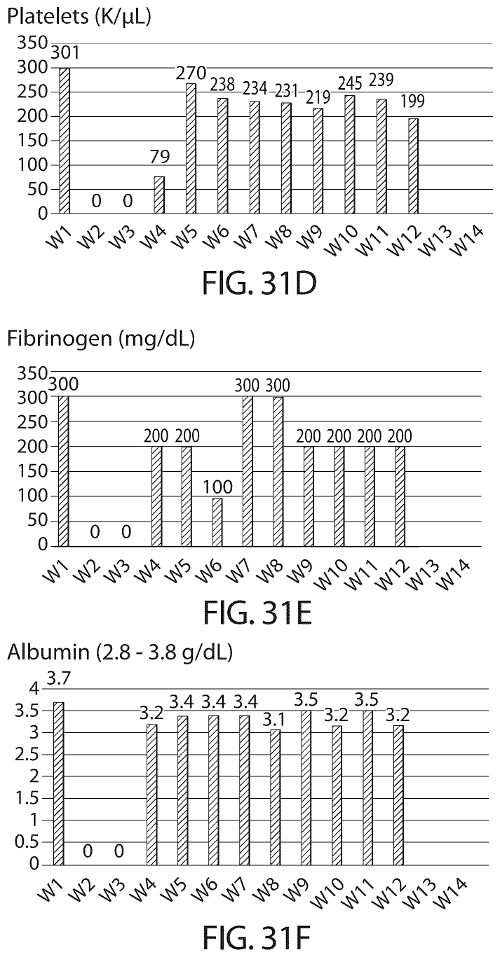

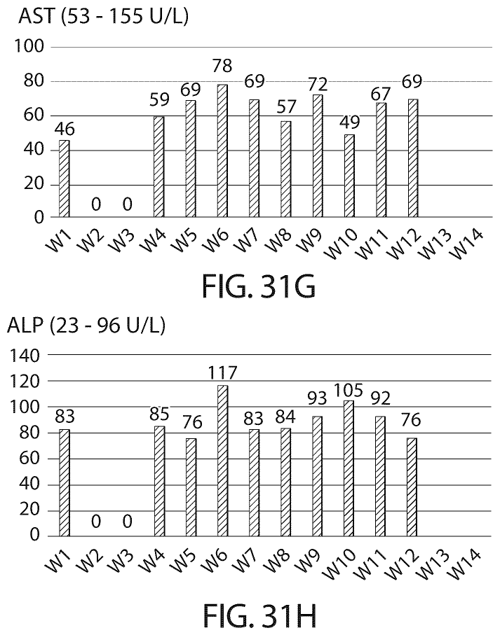

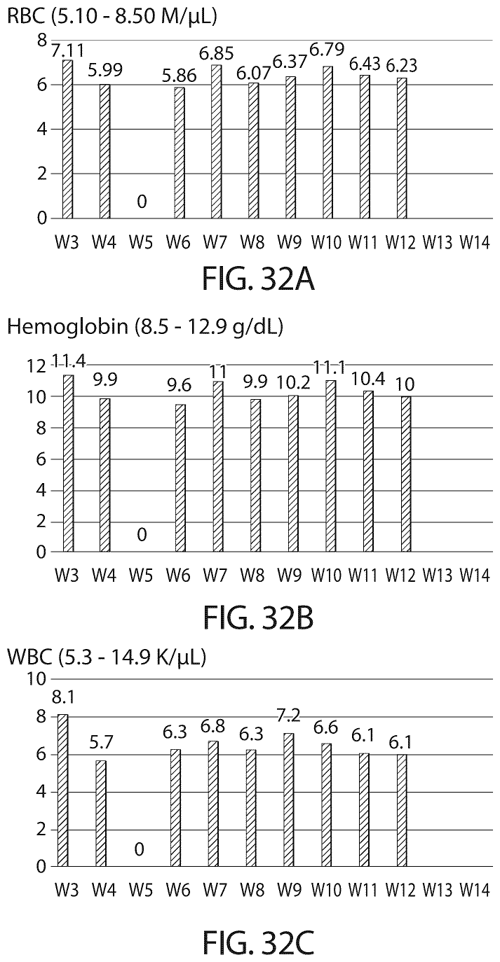

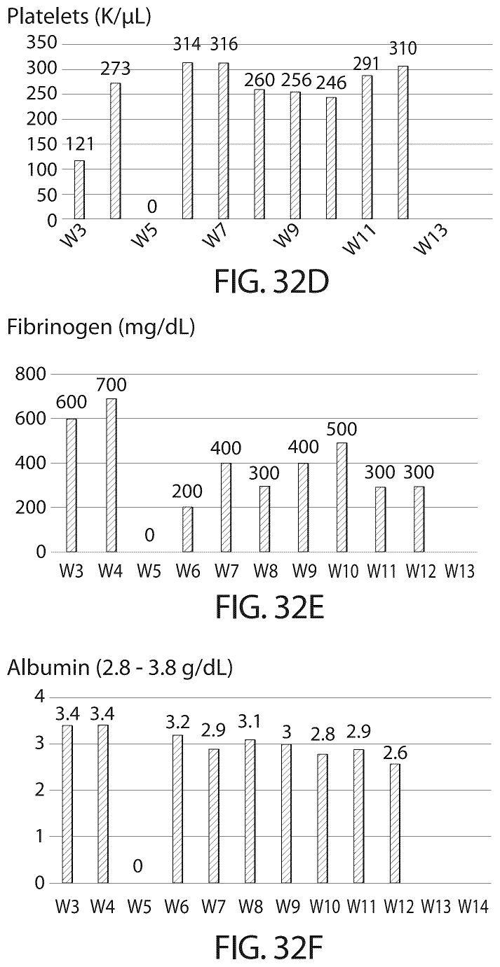

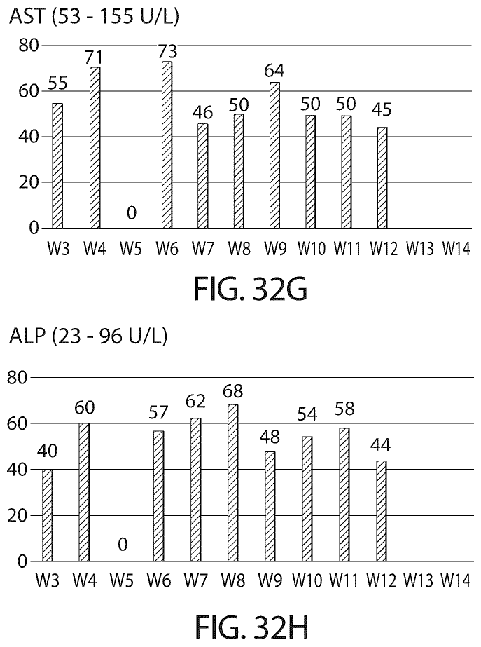

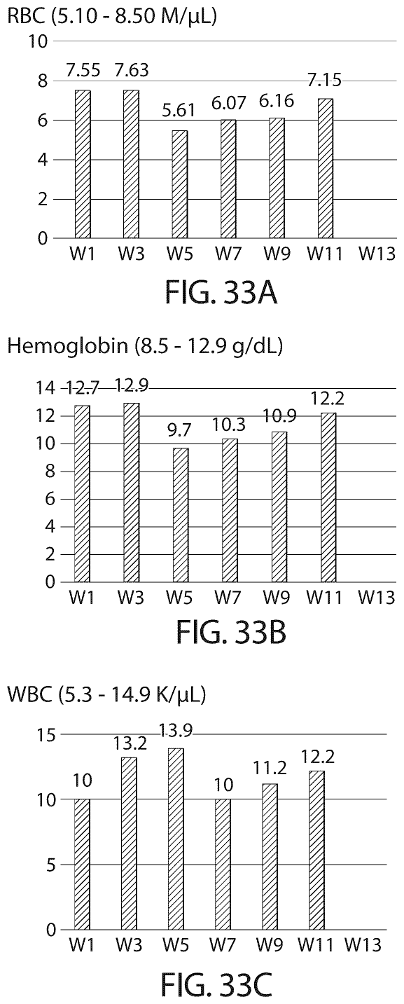

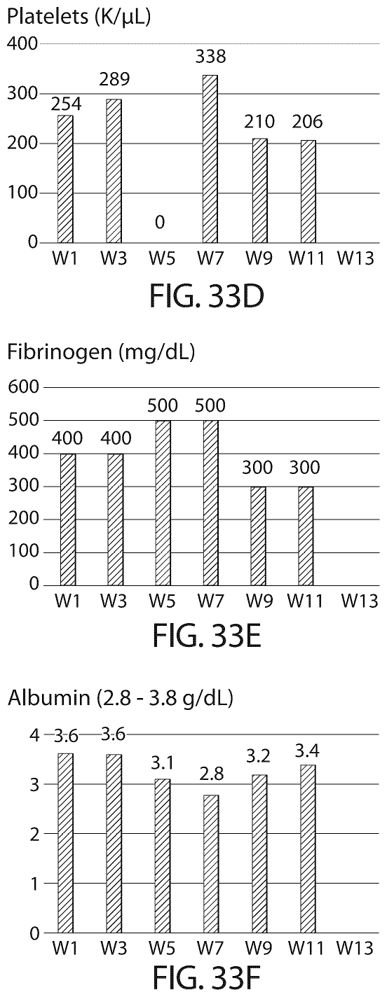

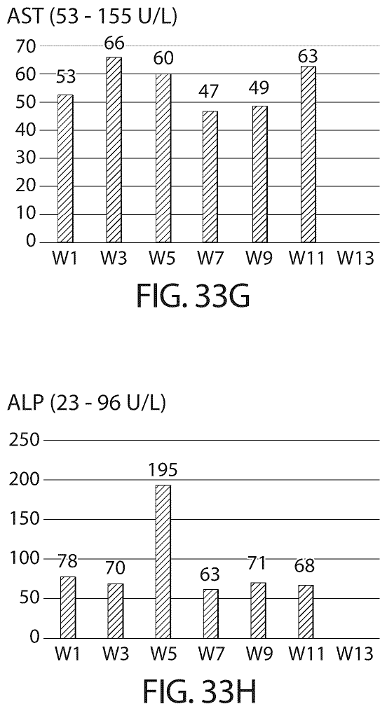

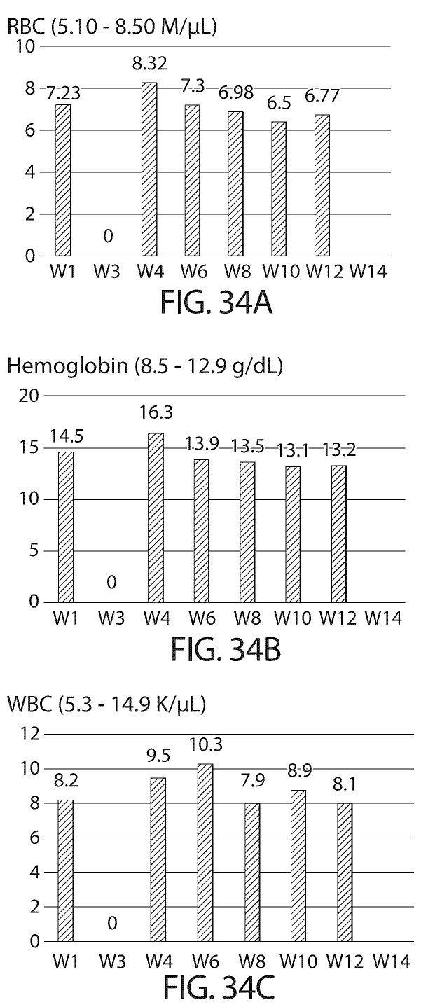

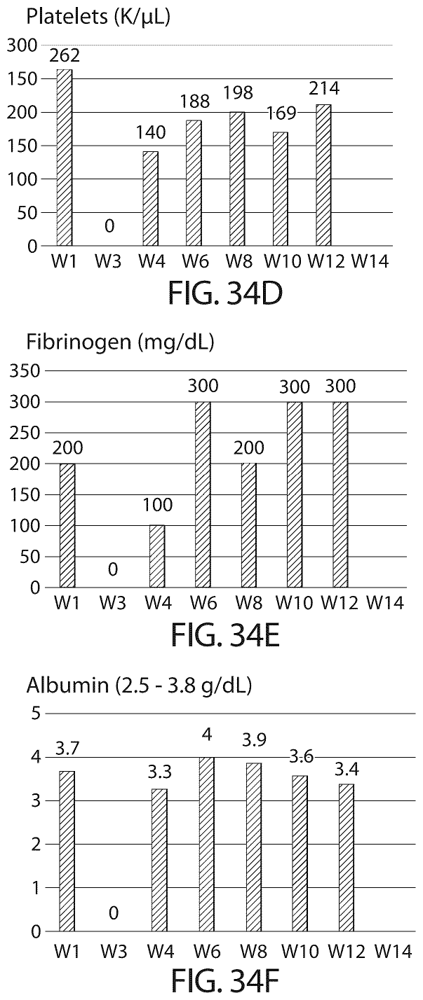

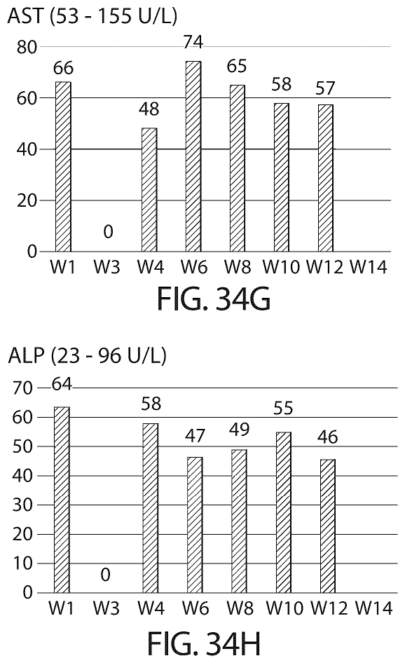

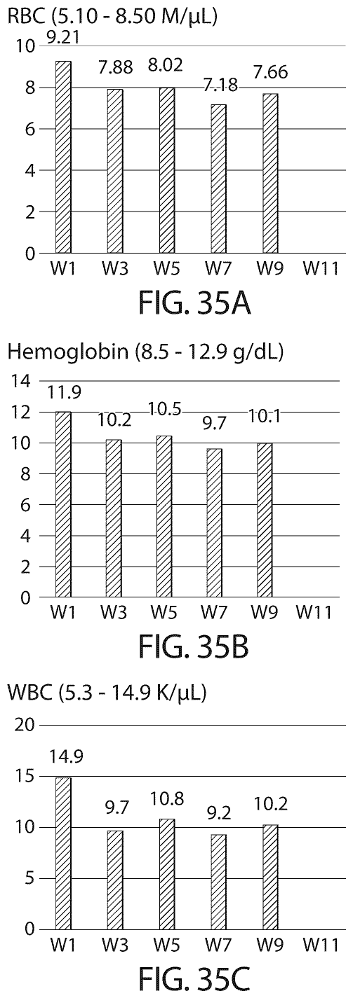

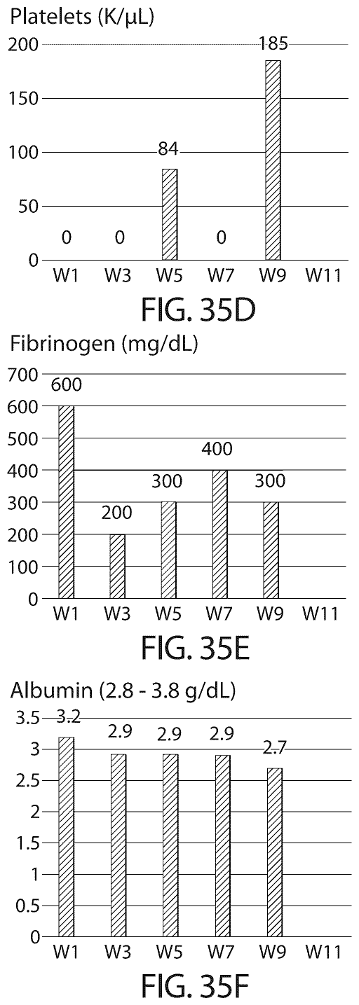

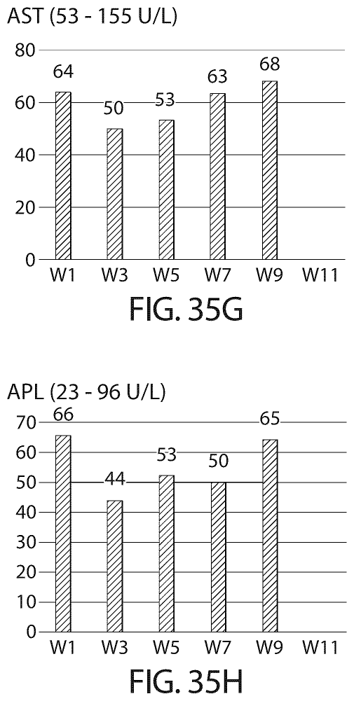

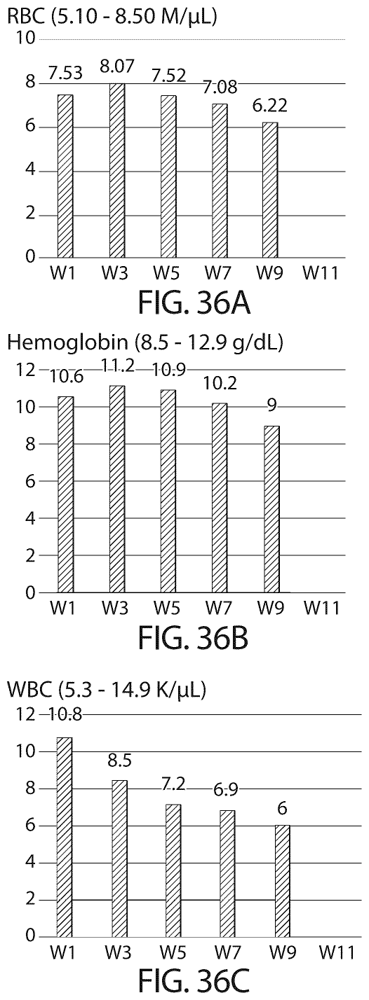

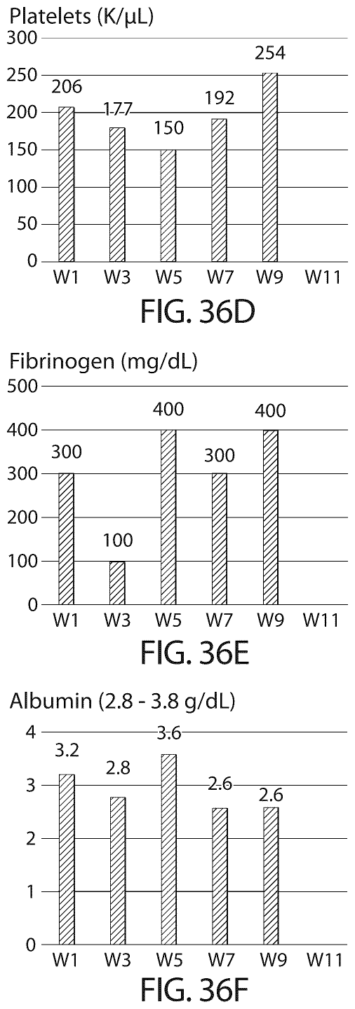

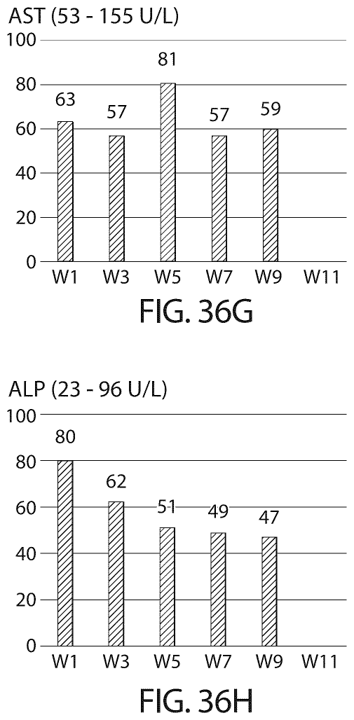

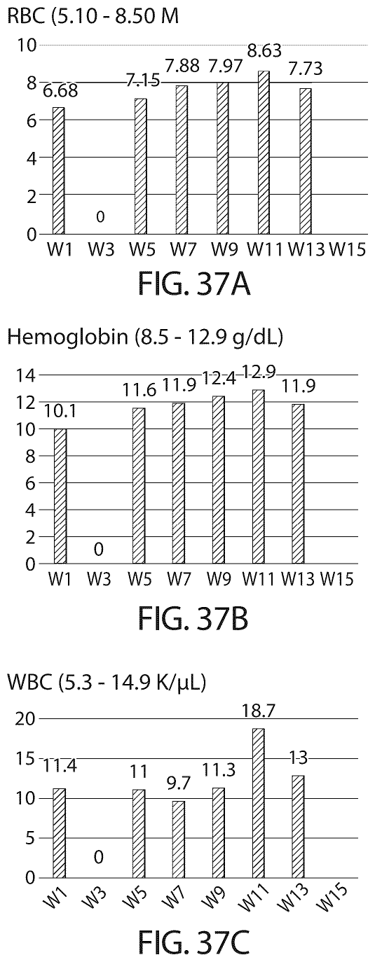

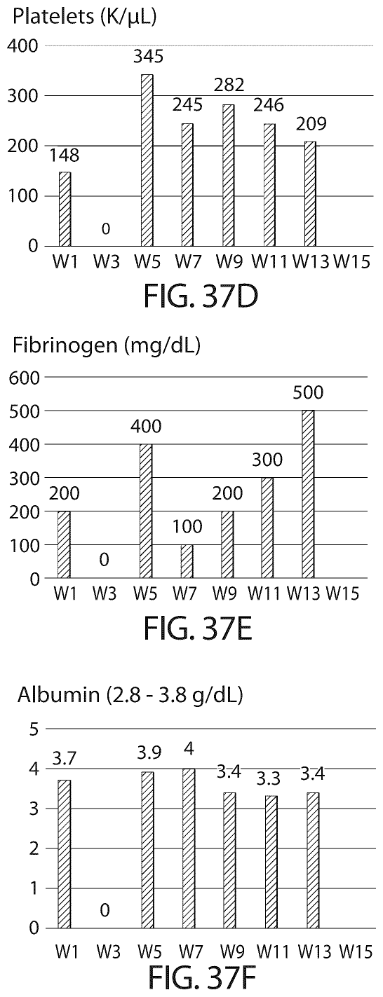

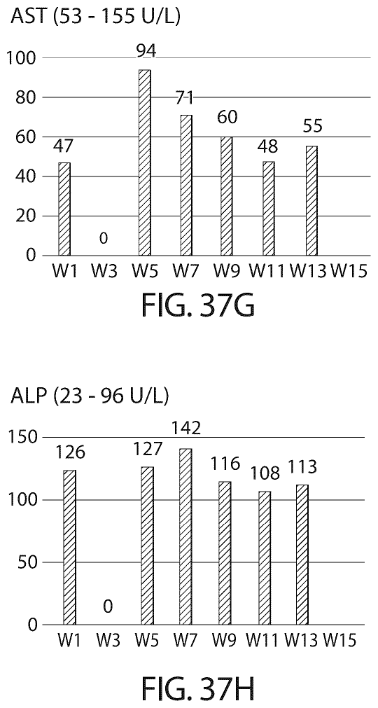

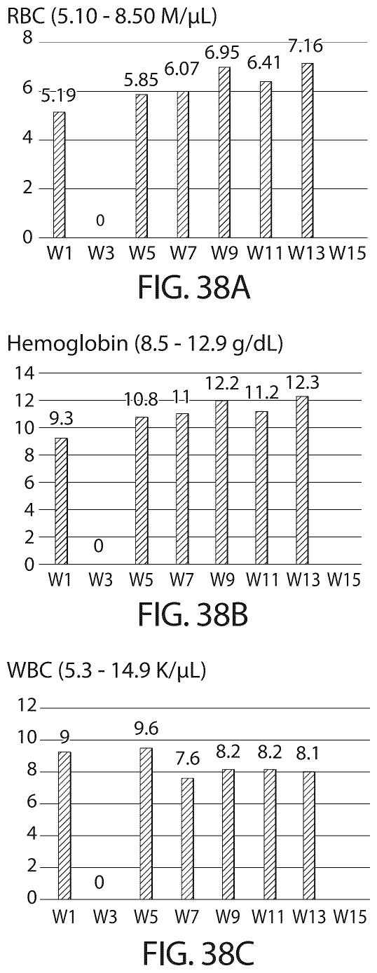

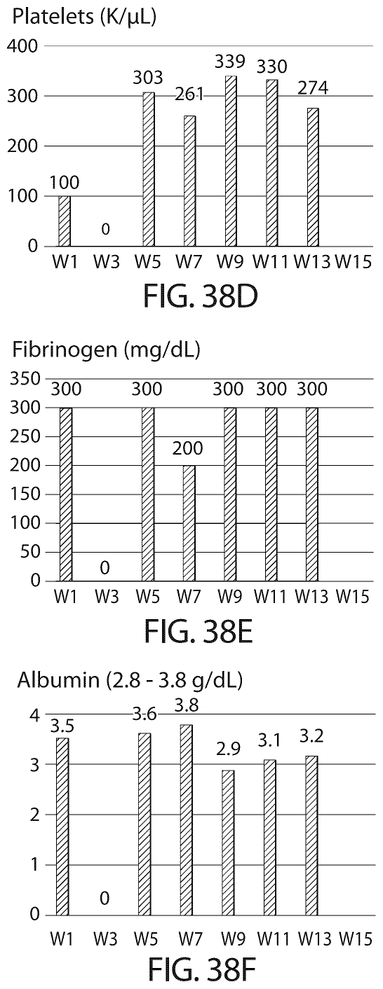

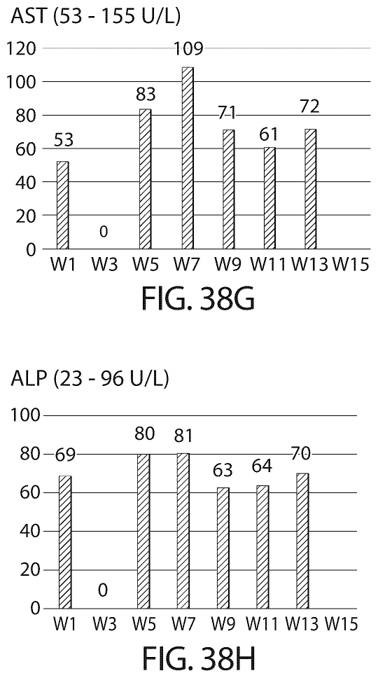

This example demonstrates the effect of repeated blood draws on the heath of the animal donors (see Table 1). Platelet-rich plasma was withdrawn either weekly on young Holstein heifers (4266, 4348) or biweekly on mature Holstein cows (2315, 2371), young steers (5211, 5276), and young Holstein heifers (14424 and 14583) for 9 weeks, 12 weeks, or 13 weeks. The total volume of platelet-rich plasma donated varied from 9 L to 14 L (for reference, the total blood volume of a cow is ˜55 mL/kg or about 33 L for a cow that weighs 1350 pounds). Whole blood draws were performed at the time of platelet-rich plasma donation and standard hematology and blood chemistry was performed. These tests are routine in veterinary medicine and provide information on the health of the animal. The tests report the red blood cell count (e.g., metric for anemia), hemoglobin levels (e.g., metric for anemia), white blood cell count (e.g., metric of infection), platelet counts (e.g., metric of clotting disorders), fibrinogen concentrations (e.g., metric of clotting disorders), albumin levels (e.g., metric of liver health), AST levels (e.g., metric of liver damage) and ALP levels (e.g., metric of liver damage). As can be seen in FIGS. 4-11, there are no observed adverse health effects from repeated platelet-rich plasma donation from young or mature Holstein cows and young steers.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The invention claimed is:

1-184. (canceled)

185. A method, comprising:
producing a plurality of meat products from one or more blood components extracted from the blood of non-human living animals, wherein the mass of the meat products is at least 110% of the mass of the non-human animals' meat after slaughtering.

186. The method of claim 185, comprising performing a blood draw on an animal, and producing one or more of the meat product from at least a portion of the blood draw.

187. The method of claim 185, comprising extracting the one or more blood components from the blood draw.

188. The method of claim 185, wherein one of the blood components comprises a platelet concentrate.

189. The method of claim 185, wherein one of the blood components comprises a platelet-rich plasma.

190. The method of claim 185, wherein one of the blood components comprises a plasma product.

191. The method of claim 185, further comprising adding the one or more blood components to a bioreactor and cultivating cells within the bioreactor to produce the meat product.

192. A method, comprising:
cultivating at least 0.1 kg of tissue/L platelet-rich plasma obtained from a first blood draw drawn from a non-human living animal; and
cultivating at least 0.1 kg of tissue/L platelet-rich plasma obtained from a second blood draw drawn from the non-human living animal.

193. The method of claim 192, further comprising withdrawing the first blood draw from the non-human living animal.

194. The method of claim 193, wherein the first blood draw has a volume of between 0.1% and 5%, in liters per kg of weight of the non-human living animal.

195. The method of claim 192, wherein the first blood draw has a volume of between 1% and 3%, in liters per kg of weight of the non-human living animal.

196. The method of claim 193, further comprising producing the platelet-rich plasma from the first blood draw using apheresis.

197. The method of claim 193, wherein the tissue is a cultivated meat product.

198. The method of claim 193, wherein the non-human living animal is a cow.

199-204. (canceled)

205. A method, comprising:
extracting blood from a non-human living animal;
isolating plasma from the blood;
producing a platelet lysate from the plasma;
producing cell culture media comprising the platelet lysate; and
culturing non-human cells in a bioreactor using the cell culture media.

206. The method of claim 250, wherein isolating plasma from the blood comprise using plasmapheresis to isolate the plasma from the blood.

207. The method of claim 250, wherein producing cell culture media comprises producing cell culture media wherein the platelet lysate is present at 2% to 20% by weight of the cell culture media.

208. The method of claim 250, wherein the non-human living animal is a cow.

209. The method of claim 250, wherein the non-human living animal and the non-human cells are from the same species.

210. The method of claim 250, further comprising introduction a plurality of microcarrier scaffolds to the bioreactor.

211. The method of claim 210, wherein the plurality of microcarrier scaffolds have an average diameter of less than 1 mm.

212. The method of claim 210, wherein the plurality of microcarrier scaffolds comprise at least 50 wt % of non-human blood plasma protein.

213. The method of claim 210, wherein the plurality of microcarrier scaffolds comprise platelet rich plasma.
